(19) United States
(12) Patent Application Publication
(10) Pub. No.: US 2010/0130457 A1
(43) Pub. Date: May 27, 2010

(54) Phenothiazine Derivatives for Treatment of Asthma

(75) Inventors: Stephen Roth, Gladwyne, PA (US); David Zope, Wayne, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Immune Control, Inc.

(21) Appl. No.: 12/618,216

(22) Filed: Nov. 13, 2009

Related U.S. Application Data
(60) Provisional application No. 61/114,741, filed on Nov. 14, 2008.
(60) Provisional application No. 61/143,549, filed on Jan. 9, 2009.
(60) Provisional application No. 61/157,074, filed on Mar. 3, 2009.

(51) Int. Cl. A61K 31/56; A61K 31/5415; A61K 31/397; A61P 37/08; A61P 11/06

(52) U.S. Cl. 514/171; 514/225.8; 514/225.5; 514/210.21

(57) ABSTRACT

The present invention relates to decreasing inflammation and airway obstruction in asthma. The invention also provides compositions and methods of reducing allergic reactions associated with asthma.

### CROSS-REFERENCE TO RELATED APPLICATIONS

This application claims priority under 35 U.S.C. §119(e) to U.S. Provisional Application No. 61/114,741, filed Nov. 14, 2008, U.S. Provisional Application 61/143,549, filed Jan. 9, 2009, and U.S. Provisional Application No. 61/157,074, filed Mar. 3, 2009, all of which are hereby incorporated by reference in their entirely herein.

### BACKGROUND OF THE INVENTION

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors” or “5-HTRs”). Classically, members of the serotonin receptor family have been grouped into seven (7) subtypes pharmacologically, i.e., according to their operational, structural, and transductional property (Hoyer et al., 2002 Pharmacol Biochem Behav Molecular 71: 533-54). Thus, while all the 5-HT receptors specifically bind with serotonin, they are pharmacologically distinct and are encoded by separate genes. To date, fourteen (14) mammalian serotonin receptors have been identified and sequenced. More particularly, these fourteen separate 5-HT receptors have been grouped into seven (7) pharmacological subtypes, designated 5-HT1, 5-HT2, 5-HT3, 5-HT4, 5-HT5, 5-HT6, and 5-HT7. Several of the subtypes are further subdivided such that the receptors are grouped pharmacologically as follows: 5-HT1A, 5-HT1B, 5-HT1D, 5-HT1E, 5-HT1F, 5-HT2A, 5-HT2B, 5-HT2C, 5-HT3A, 5-HT3B, 5-HT4, 5-HT5, 5-HT6, 5-HT7. The nucleic and amino acid sequences of the receptors do not predict their pharmacological groupings.

Of the fourteen different mammalian serotonin receptors that have been cloned, all but one are members of the G-protein coupled receptor superfamily. Serotonin receptors 5-HT1A, 5-HT1B, and 5-HT1D inhibit adenylate cyclase, and 5-HT2 receptors activate phospholipase C pathways, stimulating breakdown of polyphosphoinositides. The 5-HT2 receptor belongs to the family of rhodopsin-like signal transducers that are distinguished by a seven-transmembrane configuration and functional linkage to G-proteins. The 5-HT3 receptor family includes ligand-gated ion channel receptors that have four putative transmemebrane domains (TMDs).

Serotonin regulates a wide variety of sensory, motor and behavioral functions in the mammalian CNS, including behaviors such as learning and memory, sleep, thermoregulation, motor activity, pain, sexual and aggressive behaviors, appetite, neuroendocrine regulation, and biological rhythms. Serotonin has also been linked to pathophysiological conditions such as anxiety, depression, obsessive-compulsive disorders, schizophrenia, suicide, autism, migraine, emesis, alcoholism and neurodegenerative disorders. This biogenic amine neurotransmitter is synthesized by neurons of the brain stem that project throughout the CNS, with highest density in basal ganglia and limbic structures (Steinbusch, 1984, In: Handbook of Chemical Neuroanatomy 3:68-125, Bjorklund et al., Eds., Elsevier Science Publishers, B. V.).

Studies have suggested that serotonin may play a role in the immune system since data demonstrate that serotonin receptors are present on various cells of the immune system. There have been reports in the literature about the immunomodulatory effects of adding serotonin exogenously to mitogenically stimulated lymphocyte cultures. Under some circumstances, serotonin has been shown to activate T cells (Foon et al., 1976, J. Immunol. 117:1545-1552; Kut et al., 1992, Immunopharmacol. Immunotoxicol. 14:783-796; Young et al., 1993, Immunology 80:395-400), whereas other laboratories report that high concentrations of added serotonin inhibit proliferation (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271).

Of the fourteen known pharmacologically distinct serotonin receptors, lymphocytes express type 2A, type 2B, type 2C, type 6 and type 7 on resting cells (Ameisen et al., 1989, J. Immunol. 142:3171-3179; Stefulj et al., 2000, Brain, Behavior, and Immunity 14:219-224), and type 1A and type 3 receptors are up-regulated upon activation (Aune et al., 1993, J. Immunol. 151:1175-1183; Meyniel et al., 1997, Immunol. Lett. 55:151-160; Stefulj et al., 2000, Brain, Behavior, and Immunity 14:219-224).

The involvement of the 5-HT1A receptors in human and mouse T cells has also been demonstrated (Aune et al., 1990, J. Immunol. 145:1826-1831; Aune et al., 1993, J. Immunol. 151:1175-1183; Aune et al., 1994, J. Immunol. 153:1826-1831). These studies established that IL-2-stimulated human T cell proliferation could be inhibited by a blockade of tryptophan hydroxylase, i.e., the first enzyme involved in the conversion of tryptophan to serotonin, and that the inhibition could be reversed by the addition of 5-hydroxytryptophan. Furthermore, human T cell proliferation was blocked in vitro with a 5-HT1A-specific receptor antagonist. In a murine model, a type 1A receptor antagonist, but not a type 2 receptor antagonist, was able to inhibit the in vivo contact sensitivity response, but not antibody responses, to oxazalone.

PCT Publication No. WO 2002/078643 discloses a method of inhibiting the interaction of serotonin with serotonin type 2 receptors on a cell using a specific antagonist, and/or inhibiting the signal(s) transduced through the serotonin type 2 receptor to inhibit activation of T cells. PCT Publication No. WO 2003/106660 discloses the use of fluphenazine, an antagonist of 5-HT(1B/1D) and 5-HT(2C) receptors, for inhibiting proliferation and inducing cell death in lymphocytes. PCT Publication No. WO 2008/027521 discloses the use of various fluphenazine derivatives for inhibiting proliferation and inducing cell death in activated lymphocytes. PCT Publication No. WO 2006/138038 discloses methods of treating psoriasis in a human where the method comprises the intralesional administration of fluphenazine, or a derivative thereof, to a psoriatic lesion on the skin of the patient.

Asthma is a chronic respiratory disease characterized by inflammation of the airways, excess mucus production and airway hyperresponsiveness, and a condition in which airways narrow excessively or too easily respond to a stimulus. Asthma episodes or attacks cause narrowing of the airways, making breathing difficult. Asthma attacks can have a significant impact on a patient's life, limiting participation in many activities. In severe cases, asthma attacks can be life-threatening. Currently, there is no cure for asthma.

Symptoms of asthma include recurrent episodes of wheezing, breathlessness, chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

Asthma likely results from complex interactions among inflammatory cells, mediators, and other cells and tissues resident in the airway. Mast cells, eosinophils, epithelial cells, macrophage, and activated T-cells all play an important role in the inflammatory process associated with asthma (Djukanovic et al., 1990 Am. Rev. Respir. Dis. 142: 434-457). These cells can influence airway function through secretion of preformed and newly synthesized mediators which can act directly or indirectly on the local tissue.

Asthma is a complex disorder which arises at different stages in development and can be classified based on the degree of symptoms as acute, subacute, or chronic inflammatory response. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

Allergic asthma can be characterized by reversible airway obstruction, elevated levels of IgE, chronic airway inflammation, airway hyperresponsiveness to bronchoconstrictive stimuli, airway tissue remodeling, and mucus hypersecretion. The allergic inflammatory infiltrate in the airway tissue predominantly consists of eosinophils and CD4+ T-lymphocytes. It is widely accepted that type 2 T-helper (Th2) lymphocytes, which produce a limited set of cytokines including interleukin-3 (IL-3), IL-4, IL-5, IL-9, IL-10 and IL-13, play an important role in the initiation and progression of allergic asthma (Roinagnani et al., 2000 J Allergy Clin Immunol 105: 399-408). Chronic asthma appears to be driven and maintained by persistence of a subset of chronically activated memory T-cells. Besides T-lymphocytes, many other inflammatory cell-types are involved in the pathophysiology of allergic asthma such as eosinophils, mast cells, B-lymphocytes, dendritic cells, macrophages and monocytes, as well as resident airway cells such as epithelial cells and smooth muscle cells.

Chronic obstructive pulmonary disease (COPD) is a term used to classify two major airflow obstruction disorders: chronic bronchitis and emphysema. Chronic bronchitis is inflammation of the bronchial airways. The bronchial airways connect the trachea with the lungs. When inflamed, the bronchial tubes secrete mucus, causing a chronic cough. Emphysema is an overinflation of the alveoli or air sacs in the lungs.

In emphysema, the alveolar sacs are overinflated as a result of damage to the elastin skeleton of the lung. Inflammatory cells in emphysematous lung release elastase enzymes, which degrade or damage elastin fibers within the lung matrix. Emphysema has a number of causes, including smoking, exposure to environmental pollutants, alpha-one antitrypsin deficiency, and aging.

Acute exacerbation of asthma is often caused by spasm of the airways, or bronchoconstriction, causing symptoms including sudden shortness of breath, wheezing, and cough. Bronchospasm is treated with inhaled bronchodilators (anticholinergics such as ipratropium and beta-agonists such as albuterol). Patients inhale these medications into their lungs as a mist, produced by either a nebulizer or a hand-held meter dose (MDI) or dry powder (DPI) inhaler. Patients with acute episodes may also be treated with oral or intravenous steroids that serve to reduce the inflammatory response that exacerbates the condition.

Medications for the treatment of asthma are generally separated into two categories, quick-relief medications and long-term control medications. Asthma patients take the long-term control medications on a daily basis to achieve and maintain control of persistent asthma. Long-term control medications include inhaled glucocorticoids, leukotriene modifiers, mast cell stabilizers, anticholinergics, methylxanthines, antihistamines, IgE blockers, and methotrexate. The quick-relief medications include short-acting β₂-adrenoceptor agonists, adrenergic agonists, anti-cholinergics, and inhaled or systemic glucocorticoids. There are many side effects associated with these drugs, and none of the drugs alone or in combination is capable of preventing or completely treating asthma. There exists a long-felt need to develop novel compounds and therapies for treating asthma. The present invention meets these needs.

### BRIEF SUMMARY OF THE INVENTION

The present invention provides a method of rapidly treating a respiratory disease or disorder in a mammal. The method comprises administering to a mammal in need thereof a therapeutically effective amount of a compound, wherein the compound is capable of modulating activity of an immune cell and a muscle cell. The compound is formula I:

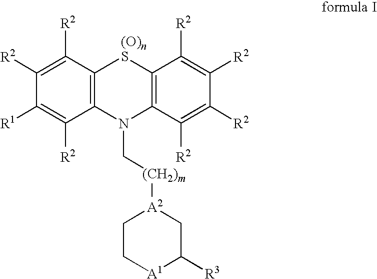

or a pharmaceutically acceptable salt, prodrug or solvate thereof, wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is CH₂, N((CH₂)ₚNR⁷₂)₂, or NR⁴;

A² is CH or N;

provided that if A¹ is CH₂, then A² is N, and if A² is CH, then A¹ is NR⁴ or N((CH₂)ₚNR⁷₂)₂

R⁴ is H, (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)ₚC(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;

R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.

R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;

R⁷ is independently selected at each occurrence from the group consisting of hydrogen and (C₁-C₆)alkyl;

R⁸ is

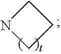

m is independently at each occurrence 1, 2, or 3;

n is 0, 1, or 2;

p is independently at each occurrence 2 or 3;

q is independently at each occurrence 1 or 2; and

t is 1, 2 or 3.

In one embodiment, the mammal is a human.

In another embodiment, the respiratory disease or condition is asthma.

In another embodiment, the compound is administered to the mammal orally, parenterally, intravascularly, intranasally, or intrabronchially.

In another embodiment, administration of the compound of the invention comprises delivery via a mist route selected from the group consisting of aerosol inhalation, dry powder inhalation, liquid inhalation, and liquid instillation.

In another embodiment, the administered compound does not substantially modulate central nervous system function of the mammal.

In another embodiment, the administered compound does not substantially cross the blood-brain barrier of the mammal.

In another embodiment, the compound inhibits proliferation of an immune cell associated with said respiratory disease or condition when the compound binds to at least 5-HT1B receptor and 5-HT7 receptor on the immune cell. In some instances, the immune cell is selected from the group consisting of a T cell, a B cell, a natural killer cell, a dendritic cell, and a macrophage, a monocyte, a neutrophil, a eosinophil, and a basophil.

In another embodiment, the compound is administered in combination with a therapeutic agent. In some instances, the therapeutic agent is an asthma/allergy medicament. In other instances, the asthma/allergy medicament is selected from the group consisting of a β₂-adrenoceptor agonist, an adrenergic agonist, a methylxanthine, an antihistamine, a prostaglandin inducer, an inhaled glucocorticoid, a systemic glucocorticoid, an immunomodulator, a leukotriene modifier, an IgE blocker, a mast cell stabilizer, an anticholinergic, a methotrexate, a PDE-4 inhibitor, a bronchodilator/beta-2 agonist, a K+ channel opener, a VLA-4 antagonist, a neurokin antagonist, a TXA2 synthesis inhibitor, a xanthanine, an arachidonic acid antagonist, a 5 lipoxygenase inhibitor, a thromboxin A2 receptor antagonist, a thromboxane A2 antagonist, an inhibitor of 5-lipox activation protein, and a protease inhibitor.

In another embodiment, the compound is administered in combination with a therapeutic agent. In some instances, the therapeutic agent is an asthma/allergy medicament. In other instances, the asthma/allergy medicament is selected from the group consisting of bronchodilator/beta-2 agonists, xanthanines, protease inhibitors, anti-histamines, steroids, prostaglandin inducers, immunomodulators, down-regulators of IgE.

In another embodiment, the therapeutic agent is administered simultaneously, prior to, or after administration of the compound of the invention.

In another embodiment, the muscle cell is selected from the group consisting of smooth muscle cell and cardiac muscle cell.

In another embodiment, the compound of the invention is administered to a mammal at a dose of about 0.01 mg/kg parenterally.

In another embodiment, the compound of the invention is administered at a dose of about 1 mg/kg parenterally.

In another embodiment, the compound of the invention is administered at a dose of about 0.1 mg/kg orally.

In another embodiment, the compound of the invention is administered at a dose of about 10 mg/kg orally.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting exemplary results from experiments of screening candidate compounds for their ability to inhibit proliferation of RPMI cells. ICI-847 and ICI-1260 have exemplary proliferation curves associated with active compounds capable of inhibiting proliferation of RPMI cells. ICI-953 has an exemplary proliferation curve associated with an inactive compound with respect to inhibiting proliferation of RPMI cells. ICI-735 is also effective, but not as effective at inhibiting proliferation of RPMI cells as are ICI-847, ICI-1260, and ICI-953.

FIG. 2 is a schematic of a murine asthma model used to test the efficacy of candidate compounds.

FIG. 3 is a chart summarizing the efficacy results from an exemplary compound for its ability to reduce methacholine-induced bronchoconstriction in the asthma animal model.

FIG. 4 is a chart depicting the total number of cells found in bronchoalveolar lavage (BAL) of asthmatic animals treated with ICI-735 and in untreated, control animals.

FIG. 5 is a chart demonstrating the selective binding of ICI-735 versus fluphenazine to different types of 5-HT receptors. ICI-735 exhibited a decreased binding to 5-HT2A receptors as compared to. In contrast, ICI-735 exhibited an increased binding to 5-HT1B receptor and 5-HT-7 receptor as compared to fluphenazine.

FIG. 6 is a chart demonstrating the pharmokinetics of ICI-735 after intraperitoneal administration (1.0 mg/kg ip and 10 mg/kg ip). The results indicate that plasma levels of ICI-735 after 1.0 mg/kg dose were comparable to levels after 10 mg/kg dose.

FIG. 7 is a chart demonstrating the oral bioavailability of ICI-735. The results indicate that plasma levels of ICI-735 after oral 5 mg/kg dose were comparable to levels after intravenous 2.5 mg/kg dose.

FIG. 8 is a chart demonstrating the effects of ICI-735 on cardiac ion channels expressed in mammalian cells. The results indicate that ICI-735 was able to block the expression of both hERG and hCaᵥ1.2 in mammalian cells.

FIG. 9 is a chart depicting the effects of ICI-735 on action potential in a mammalian cell. It was observed that ICI-735 was able to block Naᵥ1.5. The IC50 for ICI-735 was calculated to be 8.3330 μM.

FIG. 10 is a chart demonstrating the effects of ICI-735 on action potential of isolated rabbit Purkinje fibers.

FIG. 11 is a schematic of a murine asthma model used to test the efficacy of candidate compounds.

FIG. 12 is a chart summarizing the efficacy results of orally administered ICI-735 on methacholine-induced bronchoconstriction in an allergic asthma animal model.

FIG. 13 is an image depicting preparation of human precision-cut lung slices.

FIG. 14 is a chart summarizing the results of the effect of ICI-735 on human small airway contraction to IgE/anti-IgE.

FIG. 15 is a chart demonstrating that ICI-735 blocks histamine induced contraction.

### DETAILED DESCRIPTION OF THE INVENTION

The present invention provides a method of rapidly treating a respiratory disorder, including but not limited to, asthma, chronic obstructive pulmonary disease

(COPD), conditions associated with bronchoconstriction, and the like. The present invention also provides a method of rapidly treating a respiratory disorder comprising administering a composition of the invention to a mammal in need thereof. Preferably, the mammal is a human.

The invention further comprises compounds and methods of rapidly treating asthma. Asthma is believed to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness, and 3) airway inflammation. Certain cells are critical to the inflammatory reaction of asthma and they include T cells, antigen presenting cells, B cells that produce IgE, mast cells, basophils, eosinophils, and other cells that bind IgE. These cells accumulate at the site of allergic reaction in the airways and release toxic products that contribute to the acute pathology and eventually to tissue destruction related to the disorder. Other resident cells, such as smooth muscle cells, lung epithelial cells, mucus-producing cells, and nerve cells may also be abnormal in individuals with asthma and may contribute to its pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic and disabling disorder requiring long-term management.

Accordingly, the invention provides methods and compositions for the prevention and/or rapid treatment of asthma. The invention is based, in some aspects, on the finding that the compounds of the invention alleviate symptoms of asthma. In some instance, the compound of the invention reduce lung resistance in the lung of an animal having asthma.

The invention also provides a method of rapidly treating asthma. Preferably, the rapid treatment of asthma is effected by administering a compound of the invention to the mammal in need thereof. In some instances, the rapid treatment of asthma is effected by administering a compound of the invention to the mammal in need thereof at a low dose. This is because the invention is partly related to the discovery that the compounds of the invention can regulate both immune and muscle cells. In some instances, muscle cells include, but are not limited to smooth muscle cells and cardiac muscle cells.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

By T cell “activation,” as the term is used herein, is meant that the T cell, when contacted with a compound, molecule, or cell capable of generating an immune response (e.g., a mitogen or antigen), detectably upregulates surface markers, such as CD25, i.e., the IL-2 receptor, initiates a phosphorylation cascade involving p56lck, causes the release of cytokines and interleukins, increases DNA synthesis which can be assessed by, among other methods, assessing the level of incorporation of ³H-thymidine into nascent DNA strands, and causes the cells to proliferate.

Similarly, “activation of a serotonin” receptor, as used herein, means that binding of serotonin with a serotonin receptor on a cell induces the typical cascade of intra and extracellular events associated with such binding.

As used herein, to “alleviate” a disease means reducing the severity of one or more symptoms of the disease.

The term “apoptosis,” as used herein, means an active process, involving the activation of a preexisting cellular pathway, induced by an extracellular or intracellular signal, causing the death of the cell. In particular, the cell death involves nuclear fragmentation, chromatin condensation, and the like, in a cell with an intact membrane.

By the term “applicator,” as the term is used herein, is meant any device including, but not limited to, a hypodermic syringe, a pipette, an iontophoresis device, a patch, and the like, for administering the compound of the invention to a mammal.

The term “airway”, as used herein, means part of or the whole respiratory system of a subject that is exposed to air. The airway includes, but is not limited to throat, tracheobronchial tree, nasal passages, sinuses, and the like. The airway also includes trachea, bronchi, bronchioles, terminal bronchioles, respiratory bronchioles, alveolar ducts, and alveolar sacs.

The term “airway inflammation”, as used herein, means a disease or condition related to inflammation on airway of subject. The airway inflammation may be caused or accompanied by allergy(ies), asthma, impeded respiration, cystic fibrosis (CF), chronic obstructive pulmonary diseases (COPD), allergic rhinitis (AR), acute respiratory distress syndrome (ARDS), microbial or viral infections, pulmonary hypertension, lung inflammation, bronchitis, cancer, airway obstruction, bronchoconstriction, and the like.

An “asthma/allergy medicament” as used herein is a composition of matter that reduces the symptoms, inhibits the asthmatic or allergic reaction, or prevents the development of an allergic or asthmatic reaction.

Inhibition of serotonin signaling is “deleterious” to a cell, as the term is used herein, where the inhibition mediates a detectable decrease in the viability of the cell. Cell viability can be assessed using standard methods that are well-known in the art, including, but not limited to, assessing the level of biomolecular synthesis (e.g., protein synthesis, nucleic acid synthesis, and the like), trypan blue exclusion, MTT reduction, uptake of propidium iodide, exposure of phosphatidylserine on the cell surface, DNA fragmentation and/or ladder formation, and the like.

The term “bioavailability” refers to the extent to which, and sometimes the rate at which, the active moiety of a drug or metabolite enters systemic circulation, thereby gaining access to the site of action. Medications that are administered intravenously are considered to have 100 percent bioavailability, that is, the complete dose of the medication reaches the systemic circulation. But drugs that are administered through other routes, such as the oral route, generally do not have 100 percent bioavailability because these drugs may have various degrees of absorption. In particular it is desired to obtain quicker and larger and/or more complete uptake of the active compound, and thereby provide for a reduction of the administered dosages or for a reduction in the number of daily administrations.

Traditionally, bioavailability determination from plasma concentration-time data usually involves administering the compound to a human or other animal, withdrawing blood samples intravenously at certain times, and determining the maximum (peak) plasma drug concentration, the time at which maximum plasma drug concentration occurs (peak time), and the area under the plasma concentration-time curve (AUC). In oral dosing, the plasma drug concentration increases with the extent of absorption; the peak is reached when a “pseudo-equilibrium” exists between the drug elimination rate and the absorption rate. Because drug elimination begins once the drug enters the bloodstream, determining bioavailability solely based on peak plasma concentration may be misleading. Peak time is also used as an index for absorption rate, because slower absorption rates result in later peak times. Therefore, researchers often select AUC as a more reliable measure of bioavailability.

A “disease” is a state of health of an animal wherein the animal cannot maintain homeostasis, and wherein if the disease is not ameliorated, the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By the term “does not substantially cross the blood-brain barrier”, as used herein, means that the inhibitor does not detectably cross the blood-brain barrier as assessed using standard assays such as those disclosed herein, known in the art, or such assays as are developed in the future to determine the permeability of a compound across the blood-brain barrier. Such assays include, but are not limited to, assessing the neuro-psychotropic effects of the compound when administered to an animal. Further, the assays encompass, among other things, assessing the concentration of the compound beyond the barrier, or an art-recognized model of the blood-brain barrier, over time to determine the permeability of the compound through the barrier.

It would be understood by the artisan that an inhibitor can be ab initio impermeable and not cross the blood-brain barrier at a detectable level. Further, it would be understood that an inhibitor of interest can be modified, using techniques well-known in the art, such that it does not detectably cross the blood-brain barrier, or crosses it at a detectably lower level that it did before it was modified. In both instances, whether it loses its ability to cross the blood-brain barrier at a detectable level or loses the ability to cross it at a lower level than before it was modified, the compound is considered to “not substantially cross the blood-brain barrier” for purposes of this section.

“An effective amount” as used herein, means an amount that provides a therapeutic or prophylactic benefit.

The term “therapeutically effective amount” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, and the like.

“Immune cell,” as the term is used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

By the term “an inhibitor of the interaction of serotonin with a serotonin receptor,” as used herein, is meant any compound or molecule that detectably inhibits signaling via a serotonin receptor. Such compounds include a serotonin receptor ligand, an inverse agonist, and the like.

“Instructional material,” as that term is used herein, includes a publication, a recording, a diagram, or any other medium of expression which can be used to communicate the usefulness of the nucleic acid, peptide, and/or compound of the invention in the kit for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

As used herein, “lung resistance” refers to a characteristic of lung function. In some instance, lung resistance measures can be calculated to determine airway function. A classic approach to assessment of lung mechanics in animals is the measurement of dynamic lung resistance (R_{L}).

As used herein, a “low dose” of the compound of the invention is less than 2.0 g/day. In some embodiments, a “low dose” ranges from about 0.5 to 1.75 g/day (e.g., 0.5, 0.75, 1.0, 1.25, 1.5 and 1.75 g/day). These doses correspond to 5-39 mg/kg/day, depending on patient body mass, including 5 to 11, 5 to 17, 5 to 22, 5 to 28, and 5 to 33 mg/kg/day. In some embodiments, a “low dose” is about 5, 6, 7, 8, 9, 10, 11, 12, 13, 14, 15, 16, 17, 18, 19, 20, 21, 22, 23, 24, 25, 26, 27, 28, 29, or 30 mg/kg/day. In other embodiments, a low dose is in the range of about 10-15 mg/kg/day.

In some embodiments, a “low dose” of the compound of the invention is 1.0 g/day or less. In some embodiments, a “ low dose” ranges from about 100 to 500 mg/day (e.g., 100, 125, 150, 175, 200, 225, 250, 300, 350,400 and 500 mg/day). These doses correspond to about 1 to 11 mg/kg/day, depending on patient body mass, including 1 to 2, 1 to 3, 1 to 4, 1 to 5, 1 to 6, 1 to 7, 1 to 8, 1 to 9, and 1 to 10 mg/kg/day. In some embodiments, a low dose is about 1, 2, 3, 4, or 5 mg/kg/day.

In some embodiments, a “low dose” of the compound of the invention is 0.5 g/day or less. In some embodiments, a “low dose” ranges from about 5 to 100 mg/day, (e.g., 5, 10, 20, 25, 30, 40, 50, 60, 70, 75, 80, 90, and 100 mg/day). These doses correspond to about 0.05 to 2.2 mg/kg/day, depending on patient body mass, including 0.05 to 0.10, 0.05 to 0.20, 0.05 to 0.30, 0.05 to 0.50, 0.05 to 0.70, 0.05 to 0.90. 0.05 to 1.10, 0.05 to 1.30, 0.05 to 1.50, 0.05 to 1.80, and 0.05 to 2.00 mg/kg/day. In some embodiments, a low dose is about 0.1, 0.2, 0.3, 0.4, 0.5, 0.6, 0.7, 0.8, 0.9, or 1.0 mg/kg/day.

By the term “modulating” an immune response, as used herein, is meant mediating a detectable increase or decrease in the level of an immune response in a mammal compared with the level of an immune response in the mammal in the absence of a treatment or compound, and/or compared with the level of an immune response in an otherwise identical but untreated mammal. The term encompasses perturbing and/or affecting a native signal or response thereby mediating a beneficial therapeutic response in a mammal, preferably, a human.

By the term “modulating” central nervous system function, as used herein, is meant mediating a detectable increase or decrease in the function of the central nervous system in a mammal compared with the level of central nervous system function in the mammal in the absence of a treatment or compound, and/or compared with the level of central nervous function in an otherwise identical but untreated mammal. The term encompasses perturbing and/or affecting a native signal or response thereby mediating a beneficial therapeutic response in a mammal, preferably, a human. In some instances, modulating central nervous system function is associated with modulating the activity of cells of the central nervous system. An example of a cell of the central nervous system is a neuron.

As used herein, “rapid treatment” or “rapidly treat” refers to any period of time up to and including any period of time up to and including about 8 hours, about 7 hours, about 6 hours, about 5 hours, about 4 hours, about 3 hours, about 2 hours, about 1 hour, about 40 minutes, about 20 minutes, about 10 minutes, about 5 minutes, about 1 minute, or about 30 seconds, and any and all whole or partial increments there between after drug administration wherein treatment is effected.

A “receptor” is a compound that binds with a ligand.

The term “respiratory diseases”, as used herein, means diseases or conditions related to the respiratory system. Examples include, but not limited to, asthma, chronic obstructive pulmonary disease (COPD), airway inflammation, allergy(ies), impeded respiration, cystic fibrosis (CF), allergic rhinitis (AR), acute respiratory distress syndrome (ARDS), lung cancer, pulmonary hypertension, lung inflammation, bronchitis, airway obstruction, bronchoconstriction, microbial infection, and viral infection, such as SARS. Other respiratory diseases referred to herein include dyspnea, emphysema, wheezing, pulmonary fibrosis, hyper-responsive airways, increased adenosine or adenosine receptor levels, particularly those associated with infectious diseases, surfactant depletion, pulmonary vasoconstriction, impeded respiration, infantile respiratory distress syndrome (infantile RDS), allergic rhinitis, and the like.

A “subject having asthma” is a person with a respiratory system disorder characterized by inflammation, narrowing of the airways and increased reactivity of the airways to inhaled agents. Asthma is frequently, although not exclusively associated with atopic or allergic symptoms. An “initiator” as used herein refers to a composition or environmental condition which triggers asthma. Initiators include, but are not limited to, allergens, cold temperatures, exercise, viral infections, SO₂.

A “serotonin antagonist” is a composition of matter which, when administered to a mammal such as a human, detectably inhibits a biological activity attributable to the level or presence of serotonin.

A “serotonin receptor antagonist” is a composition of matter which, when administered to a mammal such as a human, detectably inhibits a biological activity attributable to serotonin binding to a serotonin receptor.

By the term “selective ligand,” as used herein, is meant a chemical agent that has a greater affinity for the target serotonin receptor type than for any other serotonin receptor family member. In some instances, a selective ligand has a greater affinity for a group of target serotonin receptor types. In some instances, the affinity is at least 5-fold more preferential affinity for a target serotonin receptor or a group of target serotonin receptor than other serotonin receptor family members.

A “serotonin receptor” includes a polypeptide that preferably binds with serotonin.

“Serotonin signal,” as the term is used herein, means a change in the balance of any intracellular biochemical pathway as a result of a receptor-mediated interaction with serotonin, a specific drug interaction with any serotonin-specific receptor, or both, that results in the change.

By the term “specifically binds,” as used herein, is meant a receptor that recognizes and binds serotonin family molecules present in a sample (i.e., dopaminergic proteins, adrenergic protein, histamines, melatonin, and serotonin), but does not substantially recognize or bind other molecules in the sample.

By the term “preferably binds,” as used herein, is meant to refer to a compound of the present invention having a greater affinity to a target serotonin receptor type than for any other serotonin receptor family member. In some instances, a compound of the invention can have a greater affinity for a group of target serotonin receptor types. In some instances, the affinity is at least 5-fold more preferential affinity for a target serotonin receptor or a group of target serotonin receptor than other serotonin receptor family members.

To “treat” a disease as the term is used herein, means to reduce the frequency of the disease or disorder reducing the frequency with which a symptom of the one or more symptoms disease or disorder is experienced by an animal. The terms “treat”, “treating” or “therapeutic”, as used herein, also mean a treatment which decreases the likelihood that the subject administered such treatment will manifest symptoms of disease or other conditions.

### Description

The invention provides compositions and methods for rapidly treating a respiratory disease, such as asthma. Preferably, the rapid treatment of asthma is effected by administering a compound of the invention to the mammal in need thereof. In some instances, the rapid treatment of asthma is effected by administering a compound of the invention to the mammal in need thereof at a low dose. Rapid treatment includes any period of time up to and including about 8 hours, about 7 hours, about 6 hours, about 5 hours, about 4 hours, about 3 hours, about 2 hours, about 1 hour, about 40 minutes, about 20 minutes, about 10 minutes, about 5 minutes, about 1 minute, or about 30 seconds, and any and all whole or partial increments there between after drug administration wherein treatment is effected. This is because the invention is partly related to the discovery that the compounds of the invention can regulate both immune and muscle cells. In some instances, muscle cells include, but are not limited to smooth muscle cells and cardiac muscle cells.

The present invention relates to compositions and methods of treating a respiratory disease. The method comprises administering a compound that binds to a 5-HT receptor, wherein the compound has the chemical formula disclosed herein. As demonstrated by the data presented herein, the compositions of the present invention are useful in rapidly treating an exemplary animal model of a asthma. In one embodiment, the compounds of the invention are useful in reducing lung resistance in an animal asthma model.

One of skill in the art would also appreciate, based upon the disclosure provided herein, that the invention encompasses rapidly treating a respiratory disease using the compositions of the invention, wherein the composition does not have a substantial effect on central nervous system function. This is because the compositions of the invention do not substantially cross the blood-brain barrier. One skilled in the art would understand that because serotonin receptors are found on neural cells and on cells of the immune system, it is desirable, but not necessary, to inhibit signaling via serotonin receptor or serotonin-like receptor on an immune cell while not affecting serotonin signaling via a serotonin receptor on a neural cell.

As demonstrated by the data disclosed herein, the compositions of the present invention reduce lung resistance in an animal asthma model. In addition, the compounds of the present invention modulate the levels of cytokines associated with a respiratory diseases. Examples of cytokines include, but are not limited to IL-1, IL-2, IL-4, IL-5, IL-6, IL-7, IL-10, IL-12, IL-13, IL-15, IL-18 granulocyte-macrophage colony stimulating factor (GM-CSF), granulocyte colony stimulating factor (GCSF), interferon-γ (γ-IFN), IFN-α, tumor necrosis factor (TNF), TGF-β, FLT-3 ligand, and CD40 ligand.

In one embodiment, the invention provides compositions and methods for treating pulmonary diseases by way of oral administration of the compositions of the invention. Preferably, the composition prevents hyper-responsiveness in airway and vascular smooth muscle. In one aspect, the compositions of the invention prevents bronchoconstriction in response to histamine and cholinergic challenges. In another aspect, the compositions diminish IL-13-induced hype responsiveness to cholinergics. In yet another aspect, the compositions block bronchoconstriction secondary to IgE-mediated mast cell degranulation.

### I. Compositions

The invention provides compositions for rapidly treating a respiratory disease, such as asthma. Preferably, the rapid treatment of asthma is effected by administering a compound of the invention to the mammal in need thereof. In some instances, the rapid treatment of asthma is effected by administering a compound of the invention to the mammal in need thereof at a low dose. Rapid treatment includes any period of time up to and including about 8 hours, about 7 hours, about 6 hours, about 5 hours, about 4 hours, about 3 hours, about 2 hours, about 1 hour, about 40 minutes, about 20 minutes, about 10 minutes, about 5 minutes, about 1 minute, or about 30 seconds, and any and all whole or partial increments there between after drug administration wherein treatment is effected. This is because the invention is partly related to the discovery that the compounds of the invention can regulate both immune and muscle cells. In some instances, muscle cells include, but are not limited to smooth muscle cells and cardiac muscle cells.

The present invention comprises compositions for rapidly treating a respiratory disease. One embodiment of the present invention, includes compositions which, as demonstrated by the data disclosed herein, reduce lung resistance. The compositions of the present invention include a composition of Formula I as well as the compositions disclosed below.

The present invention comprises the use of an effective amount of a compound according to formula I, including, but not limited to ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, ICI-1505, or a pharmaceutically acceptable salt thereof. Preferably, the present invention comprises the use of an effective amount of ICI-735, or a pharmaceutically acceptable salt thereof. Compounds according to formula I have the following chemical structure:

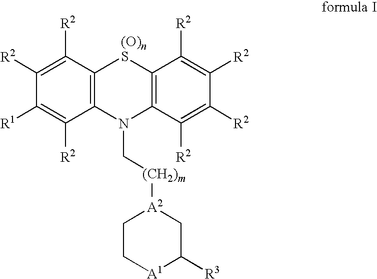

or a pharmaceutically acceptable salt, prodrug or solvate thereof, wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is CH₂, N((CH₂)ₚNR⁷₂)₂, or NR⁴;

A² is CH or N;

provided that if A¹ is CH₂, then A² is N, and if A² is CH, then A¹ is NR⁴ or N((CH₂)ₚNR⁷₂)₂;

R⁴ is H; (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;

R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.

R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;

R⁷ is independently selected at each occurrence from the group consisting of hydrogen and (C₁-C₆)alkyl;

R⁸ is

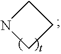

m is independently at each occurrence 1, 2, or 3;

n is 0, 1, or 2;

p is independently at each occurrence 2 or 3;

q is independently at each occurrence 1 or 2; and

t is 1, 2 or 3.

In the definitions of each of the compounds of formula I above:

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tertbutyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable monounsaturated or di-unsaturated straight chain, branched chain or cyclic hydrocarbon group having the stated number of carbon atoms. Examples include vinyl, propenyl(allyl), crotyl, isopentenyl, butadienyl, 1,3-pentadienyl, 1,4-pentadienyl, cyclopentenyl, cyclopentadienyl and the higher homologs and isomers. A functional group representing an alkene is exemplified by CH═CHCH₂.

The term “alkylene”, by itself or as part of another substituent means, unless otherwise stated, a divalent straight, branched or cyclic chain hydrocarbon.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “heteroaryl” refers to a heterocycle having aromatic character. A polycyclic heteroaryl may include one or more rings that are partially saturated. Examples include tetrahydroquinoline and 2,3 dihydrobenzofuryl. For compounds of formula I, the attachment point is understood to be on an atom that is part of an aromatic monocyclic ring or a ring component of a polycyclic aromatic that is itself an aromatic ring.

Examples of heteroaryl groups include: pyridyl, pyrazinyl, pyrimidinyl, particularly 2 and 4 pyrimidinyl, pyridazinyl, thienyl, furyl, pyrrolyl, particularly 2 pyrrolyl, imidazolyl, thiazolyl, oxazolyl, pyrazolyl, particularly 3 and 5 pyrazolyl, isothiazolyl, 1,2,3 triazolyl, 1,2,4 triazolyl, 1,3,4 triazolyl, tetrazolyl, 1,2,3 thiadiazolyl, 1,2,3 oxadiazolyl, 1,3,4 thiadiazolyl and 1,3,4 oxadiazolyl.

Examples of polycyclic heterocycles include: indolyl, particularly 3,4,5,6 and 7 indolyl, indolinyl, quinolyl, tetrahydroquinolyl, isoquinolyl, particularly 1 and 5 isoquinolyl, 1,2,3,4 tetrahydroisoquinolyl, cinnolinyl, quinoxalinyl, particularly 2 and 5 quinoxalinyl, quinazolinyl, phthalazinyl, 1,8 naphthyridinyl, 1,4 benzodioxanyl, coumarin, dihydrocoumarin, benzofuryl, particularly 3,4,1,5 naphthyridinyl, 5,6 and 7 benzofuryl, 2,3 dihydrobenzofuryl, 1,2 benzisoxazolyl, benzothienyl, particularly 3,4,5,6, and 7 benzothienyl, benzoxazolyl, benzthiazolyl, particularly 2 benzothiazolyl and 5 benzothiazolyl, purinyl, benzimidazolyl, particularly 2 benzimidazolyl, benztriazolyl, thioxanthinyl, carbazolyl, carbolinyl, acridinyl, pyrrolizidinyl, and quinolizidinyl.

The aforementioned listing of heteroaryl moieties is intended to be representative and not limiting.

The term halogen means, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —CF₃.

The compounds of formula I or those disclosed herein can be prepared by a person skilled in the art of synthetic organic chemistry. The person skilled in the art knows how to select and implement appropriate synthetic routes. Suitable synthetic methods may be identified by reference to the literature describing synthesis of analogous compounds, and then performing the synthesis of the desired compound following the route used for the analogous compounds, modifying the starting materials, reagents, and reaction conditions as appropriate to synthesizing any particular desired compounds. In addition, reference may be made to sources such as Comprehensive Organic Synthesis, Ed. B. M. Trost and I. Fleming (Pergamon Press 1991), Comprehensive Organic Functional Group Transformations, Ed. A. R. Katritzky, O. Meth Cohn, and C. W. Rees (Pergamon Press, 1996), Comprehensive Organic Functional Group Transformations II, Ed. A. R. Katritzky and R. J. K. Taylor (Editor) (Elsevier, 2nd Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996), the entire disclosures of which are incorporated herein by reference.

It will be understood that when compounds of formula I or those disclosed herein contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention that are efficacious in the treatment of a respiratory disease.

The isomers resulting from the presence of a chiral center comprise a pair of non superimposable isomers that are called “enantiomers.” Single enantiomers of a pure compound are optically active, i.e., they are capable of rotating the plane of plane polarized light.

The present invention is meant to encompass diastereoisomers as well as their racemic and resolved, diastereomerically and enantiomerically pure forms and salts thereof. Diastereoisomeric pairs may be resolved by known separation techniques including normal and reverse phase chromatography, and crystallization.

By “isolated optical isomer” means a compound that has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well known chiral separation techniques. According to one such method, a racemic mixture of a compound having the structure of Formula I or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The present invention further comprises compositions for reducing lung resistance and for treating a respiratory disease. The compositions of the present invention include the compositions disclosed below.

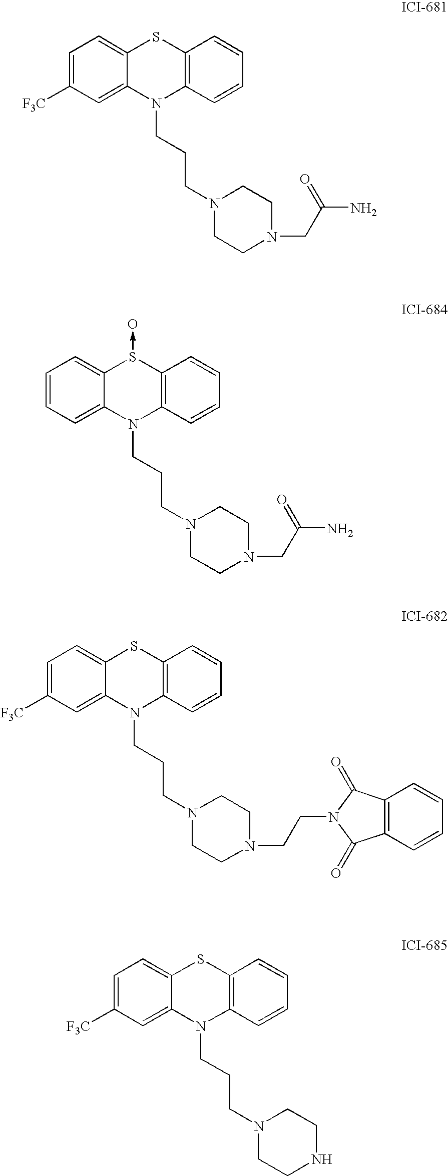

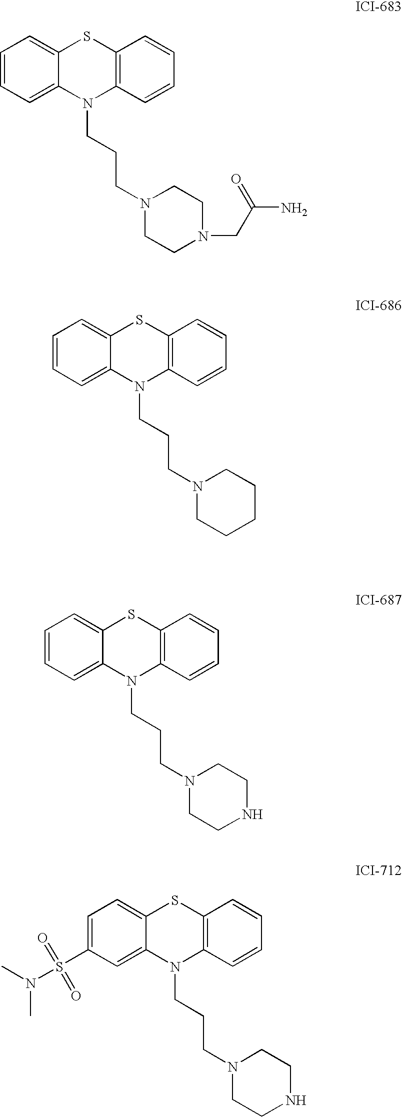

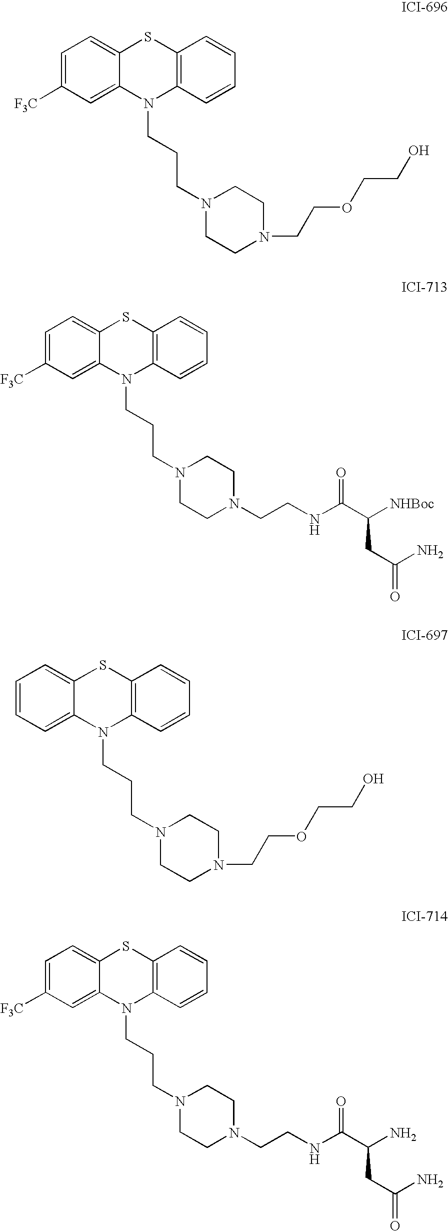

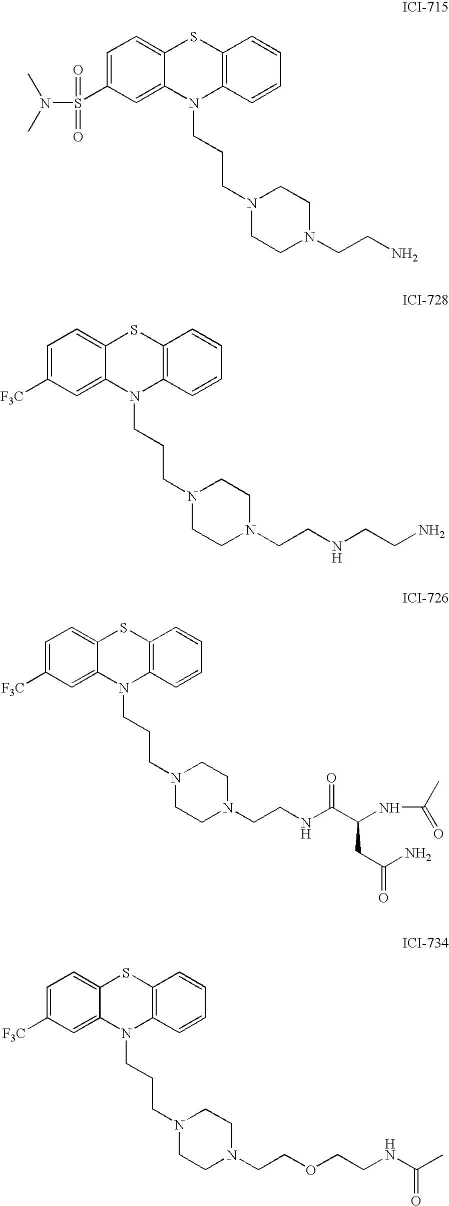

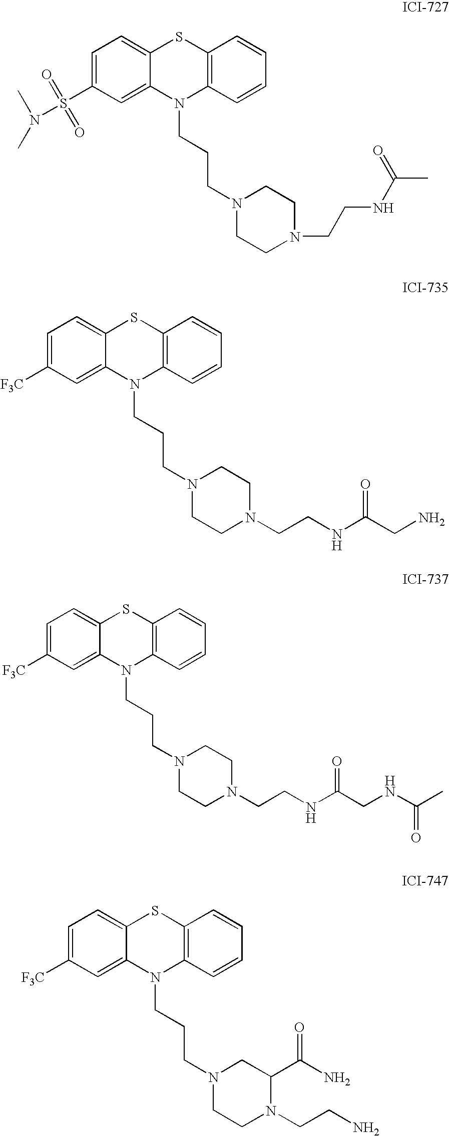

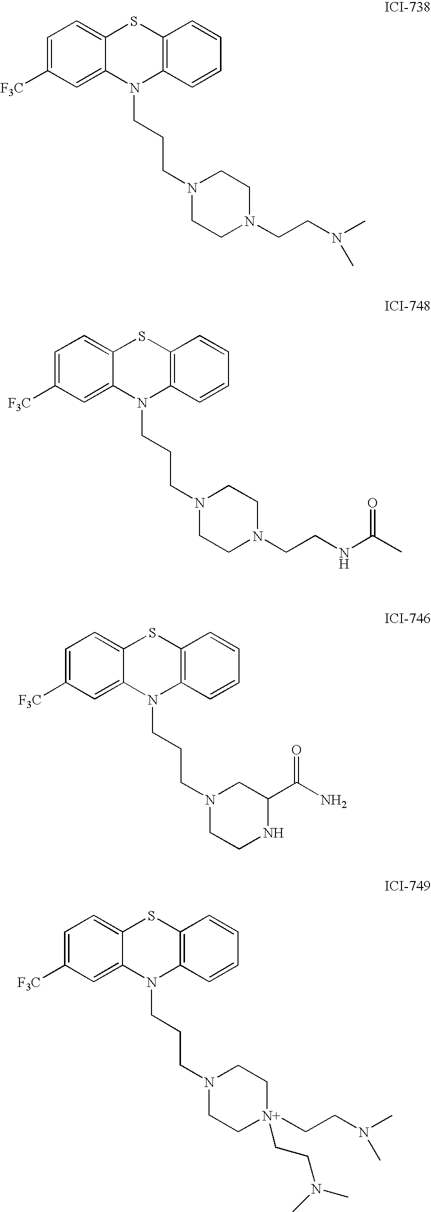

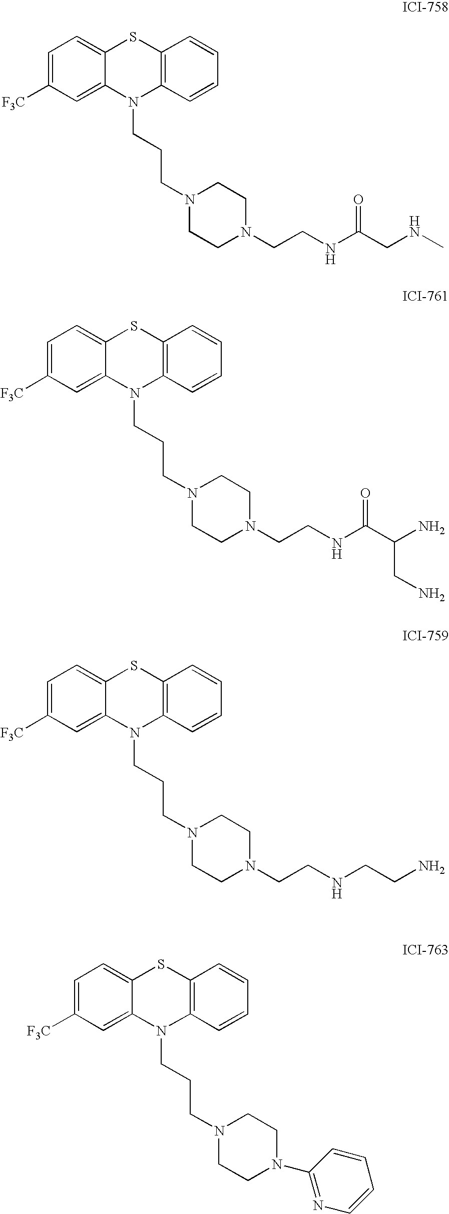

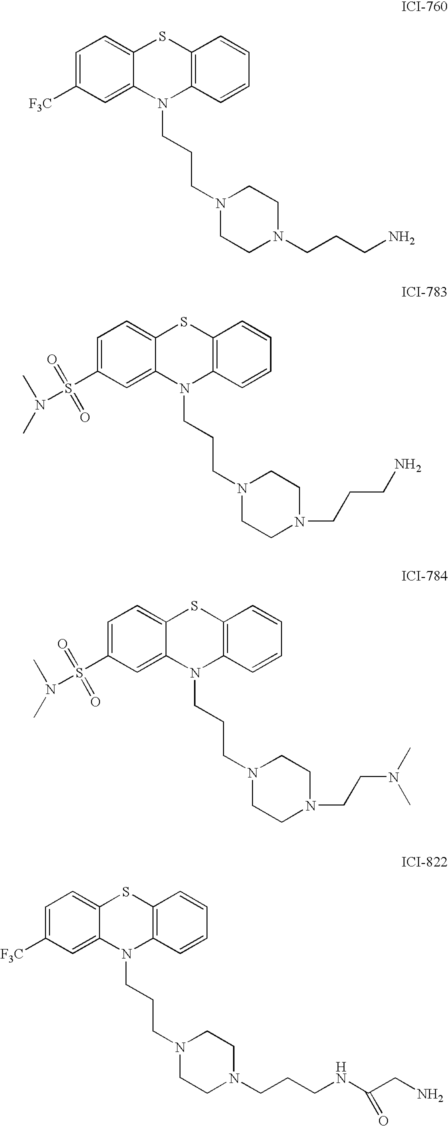

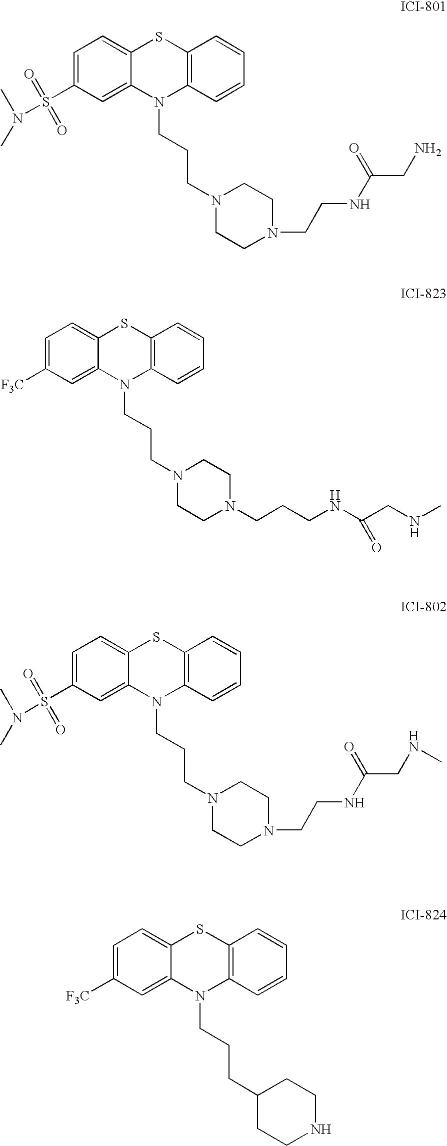

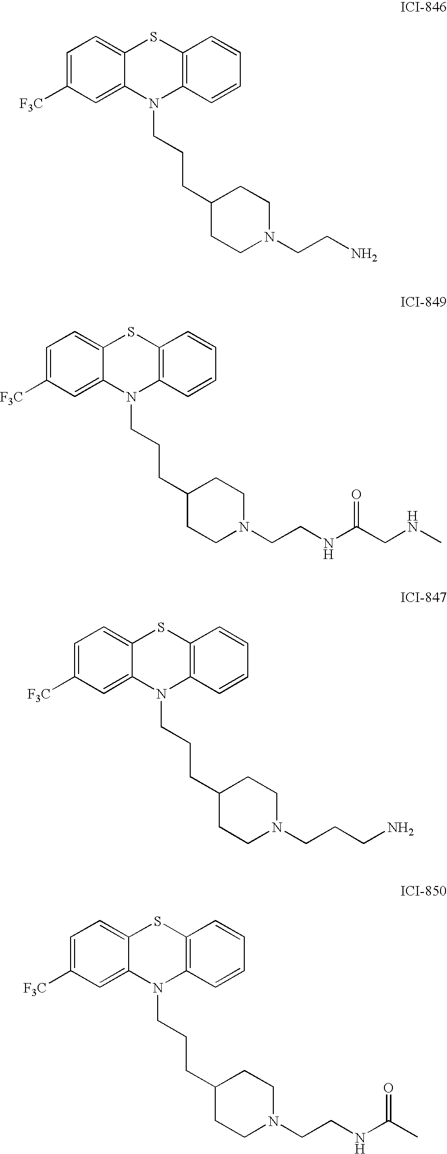

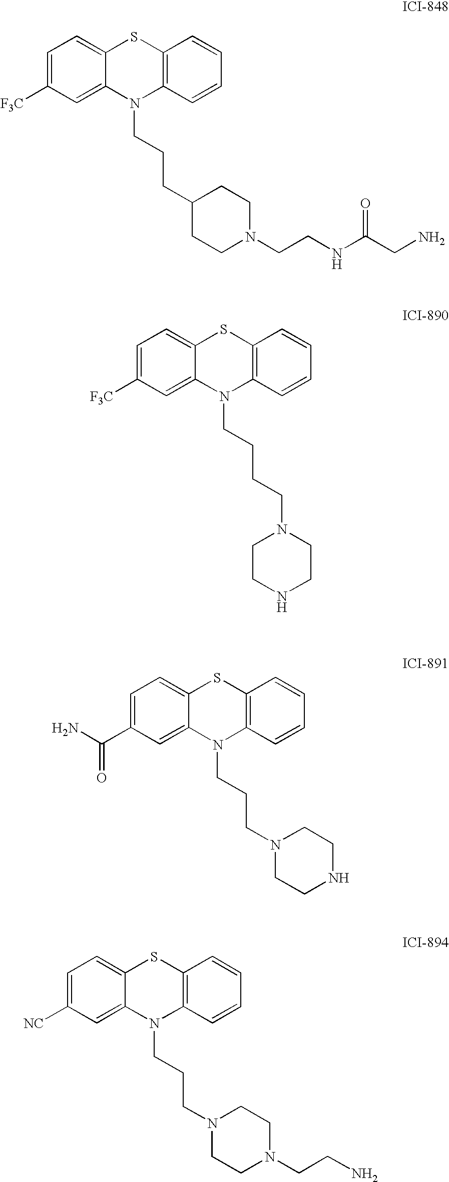

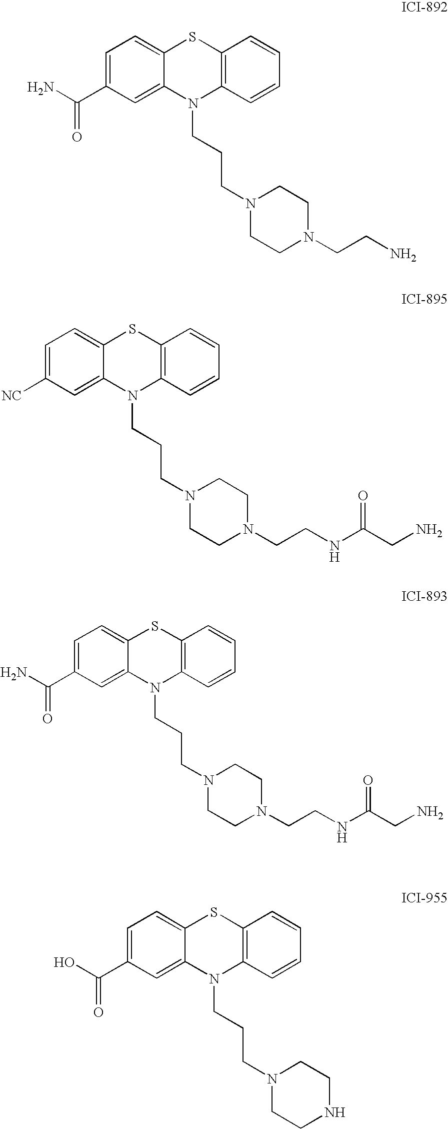

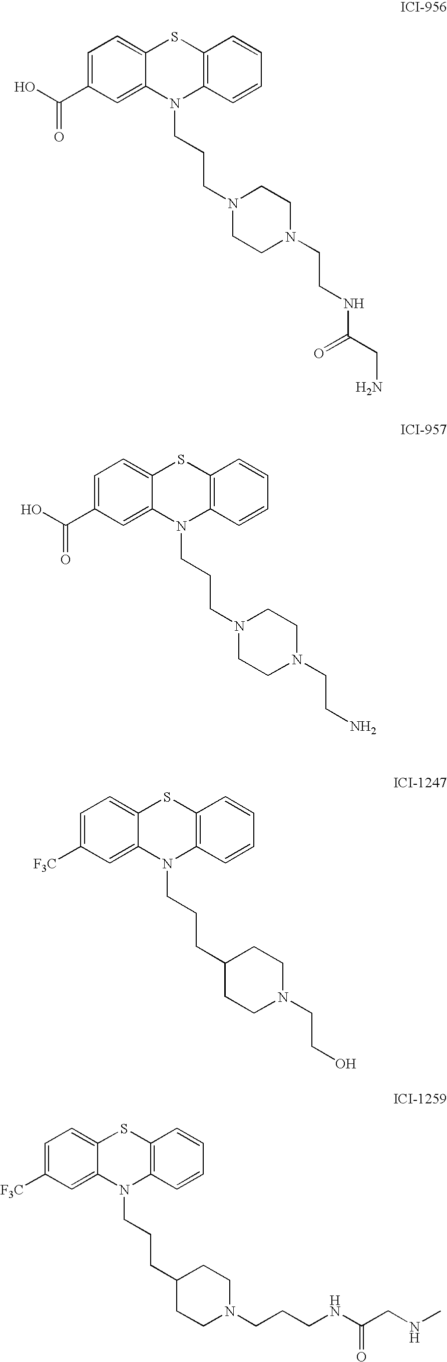

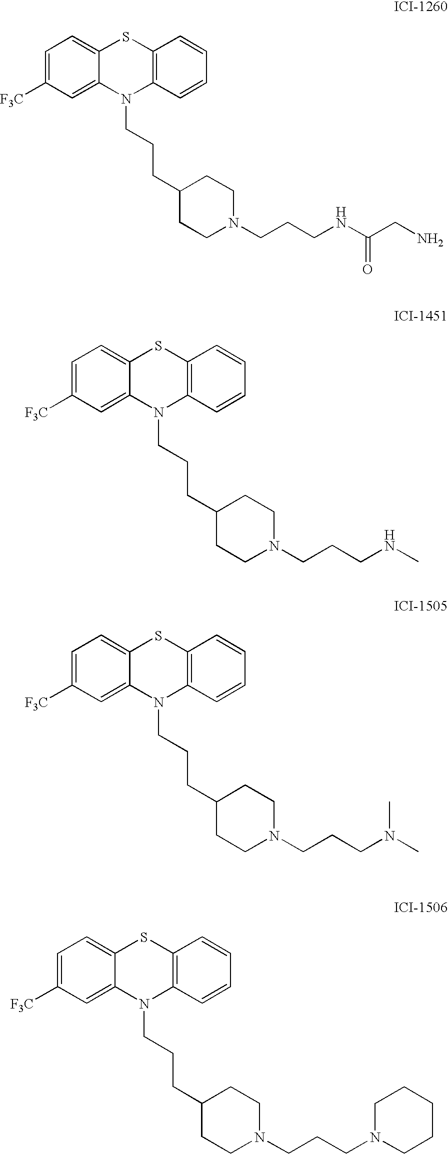

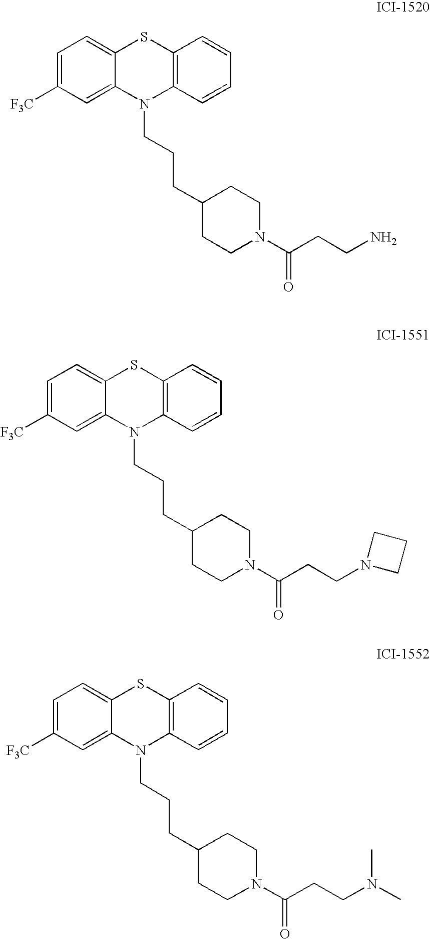

The invention is not limited to the abovementioned compounds. Rather, any compound substance that binds to a 5-HT receptor and reduces lung resistance is included in the invention. For example, the compounds of the invention in some instance can bind to receptors for hormones, neurotransmitters, growth factors, dopamine, calcitonin, adrenergic hormones, endothelin, cAMP, adenosine, acetylcholine, histamine, thrombin, kinin, follicle stimulating hormone, opsins, endothelial differentiation gene-1, rhodopsins, odorants, cytomegalovirus, phospholipase C, adenyl cyclase, phosphodiesterase, protein kinase A, protein kinase C, and the like.

One skilled in the art when armed with the present disclosure would understand that the compounds of the invention can allosterically modulate, e.g., allosterically potentiate/enhance or suppress/attenuate, the ability of the corresponding receptor to be bound by other compounds. Thus, it is contemplated that the compounds of the invention can behave as allosteric modulators of 5-HT receptor.

Accordingly, the present invention encompasses using a compound that while inhibiting serotonin signaling via a serotonin receptor or a serotonin-like receptor on a cell, does not substantially cross the blood-brain barrier. Such compounds are disclosed elsewhere herein and include the compound of formula I, as well as those disclosed elsewhere herein, but preferably includes ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, and ICI-1505.

One skilled in the art would understand, based upon the disclosure provided herein, that methods to modify a compound to affect its ability to cross the blood-brain barrier are well-known in the art, which also teaches a wide plethora of assays for assessing the ability of a substance to cross the barrier. One such method is disclosed herein, i.e., adding various side groups to a compound such as fluphenazine, thereby decreasing the ability of the compounds of the invention to cross the blood-brain barrier. The modified fluphenazine compounds, designated, e.g., formula I, are disclosed herein, but the present application is in no way limited to these or any other particular derivatives of fluphenazine. Instead, the invention encompasses any compound having the desired immunomodulatory characteristics of the inhibitors of the invention, while also possessing the desired reduced ability to cross the blood-brain barrier. The production and identification of compounds having these characteristics are routine in the art, as are assays for assessing the permeability of a compound through the blood-brain barrier. Such assays are exemplified herein, as are methods of producing compounds of interest having the desired characteristics. Nonetheless, the present invention is in no way limited to these, or any other, methods in particular; rather, it includes methods of producing and identifying compounds that do not substantially cross the blood-brain barrier and still inhibit cellular signaling via a serotonin receptor or a serotonin-like receptor such as those disclosed herein, known in the art, or to be developed in the future.

The invention is partly based on the discovery that ICI-735 was able to effectively decrease lung resistance as measured by cm H2O/ml/sec in an animal asthma model. The effectiveness of ICI-735 was at least as effective as dexamethasone, which is a well known compound used in the art for treating asthma, in alleviating a symptom of asthma in the aminal model. The effectiveness of ICI-735 in treating asthma was unexpected in view of the fact that light of ICI-735 did not inhibit proliferation of RPMI cells in vitro as well as some other compounds.

### II. Methods

The invention includes methods of rapidly treating a respiratory disease, such as asthma. Preferably, the rapid treatment of asthma is effected by administering a compound of the invention to the mammal in need thereof. In some instances, the rapid treatment of asthma is effected by administering a compound of the invention to the mammal in need thereof at a low dose. Rapid treatment includes any period of time up to and including about 8 hours, about 7 hours, about 6 hours, about 5 hours, about 4 hours, about 3 hours, about 2 hours, about 1 hour, about 40 minutes, about 20 minutes, about 10 minutes, about 5 minutes, about 1 minute, or about 30 seconds, and any and all whole or partial increments there between after drug administration wherein treatment is effected. This is because the invention is partly related to the discovery that the compounds of the invention can regulate both immune and muscle cells. In some instances, muscle cells include, but are not limited to smooth muscle cells and cardiac muscle cells.

The present invention includes a method of rapidly decreasing lung resistance in a lung that exhibits a condition of a respiratory disease. The method comprises contacting an immune cell with a compound of formula I or a compound selected from, among others, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, and ICI-1505. This is because, as demonstrated by the data disclosed herein, administration of a compound of the invention rapidly reduces lung resistance in an animal asthma model. In addition, the data disclosed herein demonstrate that the preferred compounds have an increased binding affinity to 5-HT1B and 5-HT7 receptors and a slightly decreased binding affinity to 5-HT2A receptor compared to fluphenazine.

The compounds of the invention can be used to rapidly treat, ameliorate, prevent or slow the progression of a number of respiratory diseases/conditions or their symptoms, including but not limited to, bronchiectasis, corpulmonale, pneumonia, lung abcess, acute bronchitis, chronic bronchitis, chronic obstructive pulmonary diseases, emphysema, pneumonitis, e.g., hypersensitivity pneumonitis or pneumonitis associated with radiation exposure, alveolar lung diseases and interstitial lung diseases, e.g., associated with asbestos, fumes or gas exposure, aspiration pneumonia, pulmonary hemorrhage syndromes, amyloidosis, connective tissue diseases, systemic sclerosis, ankylosing spondylitis, allergic granulomatosis, granulomatous vasculitides, asthma, e.g., acute asthma, chronic asthma, atopic asthma, allergic asthma or idiosyncratic asthma, cystic fibrosis and associated conditions, e.g., allergic bronchopulmonary aspergillosis, chronic sinusitis, inflammation or Haemophilus influenzae, S. aureus or Pseudomonas aeruginosa infection, and the like. In some of these conditions where inflammation plays a role in the pathology of the condition, the compounds of the invention can ameliorate or slow the progression of the condition by reducing damage from inflammation. In other embodiments, the compounds act to limit pathogen replication or pathogen-associated lung tissue damage. In yet other embodiments, the compounds act to reduce the immune response associate with the respiratory disease/condition.

The compounds of the invention can be used to inhibit or ameliorate one or more inappropriate immune responses or their symptoms in autoimmunity, inflammation, allergy, asthma or related conditions. The effects of the compounds include detectably ameliorating one or more of (1) the proliferation, differentiation or chemotaxis of T cells, (2) reducing unwanted cytotoxic T cell responses, (3) reducing unwanted autoantibody or other antibody synthesis, e.g., an unwanted IgA, IgE, IgG or IgM, in allergy, asthma or another autoimmune or inflammation condition, (4) inhibiting the development, proliferation or unwanted activity of autoreactive T or B cells, (5) altering the expression of one or more cytokines, interleukins or cell surface antigens, e.g., a cytokine, interleukin or cell surface antigen described herein, (6) decreasing eosinophilia in allergy conditions, (7) detectably decreasing the level or activity of one or more of ICAM-1, IL-1α, IL-1β, TNFα, IL-13, IL-4, IL-6 or IL-8 in, e.g., inflammation conditions or in autoimmune conditions, (8) decreasing the level or biological activity of one or more of TNF, IFN-γ, and IL-1, (9) reducing induction of arachidonic acid metabolism or reducing eicosanoid metabolites such as thromboxanes or prostaglandins in, e.g., asthma, (10) reducing IL-4, IL-8 or IL-10 synthesis, levels or activity in, e.g., allergy or inflammation such as idiopathic pulmonary fibrosis or allergic asthma or (11) reducing or interfering with neutrophil chemotaxis by, e.g., reducing thioredoxin release from affected cells in conditions such as cancer, infections, inflammation or autoimmunity.

In one embodiment, the invention is a method of the prophylaxis or treatment of asthma comprising administering a composition of the invention to a subject in need of such treatment, wherein the amount of the composition is sufficient for the prophylaxis or treatment of asthma in the subject.

In asthma, chronic inflammatory processes in the airway play a central role in increasing the resistance to airflow within the lungs. Many cells and cellular elements are involved in the inflammatory process, particularly mast cells, eosinophils T lymphocytes, neutrophils, epithelial cells, and even airway smooth muscle itself. The reactions of these cells result in an associated increase in the existing sensitivity and hyper-responsiveness of the airway smooth muscle cells that line the airways to the particular stimuli involved. Therefore, the invention also includes rapidly treating asthma with a compound of the invention, wherein the compound is able to regulate the biological activity of a muscle cell including, but not limited to smooth muscle cells and cardiac muscle cells.

Without wishing to be bound by any particular theory, it is believed that the ability of the compounds of the invention to regulate the biological activity of a muscle cell provides a method of treating asthma. For example, the compounds of the invention can be used to increase airway diameter. Hypertrophy of smooth muscle, chronic inflammation of airway tissues, and general thickening of all parts of the airway wall can reduce the airway diameter in patients with reversible obstructive pulmonary disease. Increasing the overall airway diameter using a variety of techniques can improve the passage of air through the airways. Application of a compound of the invention to the airway smooth muscle of an asthmatic patient can debulk or reduce the volume of smooth muscle. This reduced volume of smooth muscle increases the airway diameter for improved air exchange.

Reducing inflammation and edema of the tissue surrounding the airway can also increase the diameter of an airway. Inflammation and edema (accumulation of fluid) of the airway are chronic features of asthma. The inflammation and edema can be reduced by application of a compound of the invention to stimulate wound healing and regenerate normal tissue. Healing of the epithelium or sections of the epithelium experiencing ongoing denudation and renewal allows regeneration of healthy epithelium with less associated airway inflammation. The less inflamed airway has an increased airway diameter both at a resting state and in constriction. The wound healing can also deposit collagen which improves parenchymal tethering.

Inflammatory mediators released by tissue in the airway wall may serve as a stimulus for airway smooth muscle contraction. Therapy that reduces the production and release of inflammatory mediator can reduce smooth muscle contraction, inflammation of the airways, and edema. Examples of inflammatory mediators are cytokines, chemokines, and histamine. The tissues which produce and release inflammatory mediators include airway smooth muscle, epithelium, and mast cells. Treatment of these structures with a compound of the invention can reduce the ability of the airway structures to produce or release inflammatory mediators. The reduction in released inflammatory mediators can reduce chronic inflammation, thereby increasing the airway inner diameter, and may also reduce hyper-responsiveness of the airway smooth muscle.

Without wishing to be bound by any particular theory, it is believed that the compounds of the invention can be used to rapidly treat asthma. This is because the compounds of the invention are capable of at least modulating the immune response and modulating muscle cells. Preferably, the method of rapidly treating asthma encompasses the use of a compound of the invention at a low dose such as about 1 mg/kg/day. In some instances, the effects of the compounds can be observed as early as about 1 hour after administration to a mammal having conditions of asthma.

As used herein, “rapid treatment” or “rapidly treat” refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, about 5 minutes, about 1 minute, or about 30 seconds, and any and all whole or partial increments there between after drug administration wherein treatment is effected.

Results presented herein demonstrate that the compounds of the invention exhibit a desirable oral bioavailability at a low dose. For example, it was observed that blood levels of ICI-735 after oral 5 mg/kg dose were comparable to levels after intraperitoneal administration 2.5 mg/kg dose. Accordingly, the present invention provides different doses for administering compounds of the. Thus, invention provides a method of achieving the desired bioavailability of the active ingredient.

It is believed that plasma concentrations of a compound of the invention correlates with a therapeutic effect including, but not limited to regulating an immune cell and muscle cell in a subject at a given dose. Thus, the efficacy of the compounds of the invention, alone or in combination with another agent may be predicted by assessing the dosage relating to the area under the curve (AUC). A physician would prescribe a lower dose of a compound of the invention compared to other compounds in the art used to treat asthma in order to obtain an equivalent AUC. In some instances, the physician may prescribe at least 1% less of a compound of the invention, at least 5% less, at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80%, at least 85%, at least 90%, at least 95%, at least 99%, or any or all whole or partial increments in between.

In one embodiment, the invention is a method of the prophylaxis or treatment of COPD comprising administering a composition of the invention to a subject in need of such treatment, wherein the amount of the composition is sufficient for the prophylaxis or treatment of COPD in the subject.

In one embodiment, the invention is a method of the prophylaxis or treatment of bronchoconstriction, lung inflammation or lung allergy comprising administering a composition of the invention to a subject in need of such treatment, wherein the amount of the composition is sufficient for the prophylaxis or treatment of bronchoconstriction, lung inflammation or lung allergy in the subject.

The allergic reaction in humans and animals has been extensively studied and the basic immune mechanisms involved are well known. Allergic conditions or diseases in humans include but are not limited to eczema, allergic rhinitis or coryza, hay fever, conjunctivitis, bronchial or allergic asthma, urticaria (hives)and food allergies; atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in humans. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

Accordingly, the compounds of the invention can be used to increase the biological clearance of allergens from tissue and mucosa. Reduced generation of the levels or activity of IgE by B cells in response to treatment with a compound of the invention and related responses is facilitated at least in part by decreased production of one or more biological response mediators, e.g., cytokines or response mediators such as protein kinase A inhibitors, substance P neuropeptide, thymus- and activation-regulated chemokine, e.g., by airway smooth muscle cells, proteinase activated receptor-2 by neurons, intracellular signal-transducing protein-6 (STAT6), Janus kinase 1, Janus kinase 6, CD40, CD86 and/or NF-kB by B cells, CD154 in T cells, and suppressor of cytokine signalling-3, phosphodiesterase 4, TNF-α, MCP-1, RANTES, CXCL10, CXCL8 (IL-8), prostaglandin E2 receptor, IL-113, IL-4, IL-5, IL-6, IL-9, IL-10, IL-13, and IL-23, by one or more other cell types such as immune cells as described herein, airway smooth muscle cells, mucosal cells or keratinocytes.

In one embodiment, the compounds of the invention can be used to reduce the symptoms, inhibit the asthmatic reaction, or prevent an allergic response. In some embodiments, the compounds of the invention are useful to some degree for treating both asthma and allergy.

The invention includes the use of the compounds of the invention in combination with other suitable therapies or therapeutic agents (e.g., asthma/allergy medicament). An “asthma/allergy medicament” as used herein is a composition of matter that reduces the symptoms, inhibits the asthmatic or allergic reaction, or prevents the development of an allergic or asthmatic reaction. Accordingly, the compounds of the invention can be used in combination with existing asthma/allergy medicaments including, but not limited to, β₂-adrenoceptor agonists, adrenergic agonists, methylxanthines, antihistamines, prostaglandin inducers, inhaled glucocorticoids, systemic glucocorticoids, immunomodulators, leukotriene modifiers, IgE blockers, mast cell stabilizers, anticholinergics, methotrexate, PDE-4 inhibitors, bronchodilator/beta-2 agonists, K+ channel openers, VLA-4 antagonists, neurokin antagonists, TXA2 synthesis inhibitors, xanthanines, arachidonic acid antagonists, 5 lipoxygenase inhibitors, thromboxin A2 receptor antagonists, thromboxane A2 antagonists, inhibitor of 5-lipox activation proteins, and protease inhibitors. Use of such combination provides an improved sustained pharmacologic effect that translates to an improved disease management. For instance, the efficacy of the combination of the compounds of the invention with an asthma/allergy medicament is improved over the use of each of the medicaments alone. In some instances, the combination of the compounds of the invention with an asthma/allergy medicament works in a synergistic manner.

Bronchodilator/β₂ adrenoceptor agonists are a class of compounds that causes bronchodilation or smooth muscle relaxation. Bronchodilator/β₂ adrenoceptor agonists include, but are not limited to, salmeterol (Serevent™, GlaxoSmithKline), salbutamol, also known as albuterol; Ventolin™/Ventorlin™, GlaxoSmithKline; Asthalin™, Cipla; Proventil™, Schering-Plough; Pro-Air™, Teva), terbutaline (Brethine™/Bricanyl™/Brethaire™), formoterol (Foradil™/Foradile™, Novartis; Oxis™, Astra Zeneca; Atock™, Astellas; Perforomist™, Dey), fenoterol (Berotec™, Boehringer-Ingelheim), bitolterol (Tornalate™, Elan Pharmaceuticals), pirbuterol (Maxair™, 3M), and orciprenaline (Alotec™/Alupent™/Metaprel™/Novasmasol™).

Long-acting β₂ adrenoceptor agonists and bronchodilators are used for long-term prevention of symptoms in addition to the anti-inflammatory therapies. They cause bronchodilation or smooth muscle relaxation, by activating adenylate cyclase, increasing cyclic AMP and producing functional antagonism of bronchoconstriction. These compounds also inhibit mast cell mediator release, decrease vascular permeability and increase mucociliary clearance. Long-acting β₂ adrenoceptor agonists include, but are not limited to, salmeterol, also known as albuterol. These compounds are usually used in combination with corticosteroids and generally are not used without any inflammatory therapy. They have been associated with side effects such as tachycardia, skeletal muscle tremor, hypokalemia, and the like. The most common combinations of inhaled steroids and long-acting bronchodilators currently in use are fluticasone/salmeterol (Advair™/Seretide™, GlaxoSmithKline), and budenoside/formoterol (Symbicort™, AstraZeneca).

Short-acting β₂ adrenoceptor agonists/bronchodilators relax airway smooth muscle, causing the increase in air flow. These types of compounds are a preferred drug for the treatment of acute asthmatic systems. Short-acting β₂ agonists include, but are not limited to, bitolterol (Tornalate™, Elan Pharmaceuticals), pirbuterol (Maxair™, 3M), and terbutaline (Brethine™/Bricanyl™/Brethaire™). Some of the adverse effects associated with the use of short-acting β₂ agonists include tachycardia, skeletal muscle tremor, hypokalemia, increased lactic acid, headache, and hyperglycemia.

Older and less selective adrenergic agonists, such as inhaled epinephrine and ephedrine tablets, have also been used in the treatment of asthma. These agents cause similar or lesser rates of cardiac side effects as albuterol. Inhaled epinephrine may be used to terminate an acute asthmatic exacerbation. These agents may be given parenterally, but adverse effects may arise from this route of administration.

Methylxanthines, including for instance theophylline (also known as dimethylxanthine; Elixophillin™/Theolair™/Theocin™/Nuelin™/Synophylate™/Bronkodyl™/Aerolate™/Theovent™), doxofylline (Maxivent™/Ansimar™/Ventax™), and aminophylline (Phyllocontin™/Truphylline™/Minomal™), have been used for long-term control and prevention of symptoms. These compounds cause bronchodilation resulting from phosphodiesterase inhibition and likely adenosine antagonism. It is also believed that these compounds may effect eosinophilic infiltration into bronchial mucosa and decrease T-lymphocyte numbers in the epithelium. Dose-related acute toxicities are a particular problem with these compounds. As a result, routine serum concentration must be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia.

Other asthma/allergy medicaments (“allergy medicaments”) can be used in combination with the compounds of the invention to treat allergy. Allergy medicaments include, but are not limited to, anti-histamines, prostaglandin inducers, and steroids.

Anti-histamines counteract histamine released by mast cells or basophils. These compounds are well known in the art and commonly used for the treatment of allergy. Anti-histamines include, but are not limited to, loratidine (Claritin™/Claritin-D™, Schering-Plough; Alavert™, Wyeth), cetirizine (Zyrtec™/Reactine™, Pfizer) and analogues, buclizine (Vibazine™/Histabutizine™/Buclifen™/Buclodin™/Longifene), fexofenadine (Allegra™/Telfast™, Sanofi-Aventis), terfenadine (Seldane™, Schering-Plough, desloratadine (NeoClarityn™/Claramax™/Clarinex™/Aerius™, Schering-Plough), norastemizole (Soltara™, Sepracor), epinastine (Elestat™/Relestat™, Allergan), ebastine (Kestine™/Evastin™/Ebastel™/Aleva™, Pharmacare), astemizole (Hismanal™, Janssen), levocabastine (Livostin™, Janssen), azelastine (Astelin™, Meda), tranilast (Rizaben™), mizolastine (Mizollen™), betahistine (SERC™), and the like.

Prostaglandin inducers are compounds that induce prostaglandin activity, regulating smooth muscle relaxation. An example of a prostaglandin inducer is rebamipide.

The asthma/allergy medicaments useful in combination with the compounds of the invention also include steroids and immunomodulators. Glucocorticoids are a class of steroid hormones characterised by an ability to bind with the glucocorticoid receptor (GR) and trigger similar effects. Glucocorticoids are distinguished from mineralocorticoids and sex steroids by their specific receptors, target cells, and effects. In technical terms, corticosteroid refers to both glucocorticoids and mineralocorticoids (as both are mimics of hormones produced by the adrenal cortex), but is often used as a synonym for glucocorticoid. In this document, glucocorticoid and corticosteroid are used interchangeably.

Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation. They are also believed to reverse β₂-receptor downregulation and to inhibit microvascular leakage.

Cortosteroids are used generally for moderate to severe exacerbations to prevent the progression, reverse inflammation and speed recovery from the disease. Cortosteroids are associated with reversible abnormalities in glucose metabolism, increased appetite, fluid retention, weight gain, mood alteration, hypertension, peptic ulcer, and rarely asceptic necrosis of femur. These compounds are useful for short-term (e.g., 3-10 days) prevention of the inflammatory reaction in inadequately controlled persistent asthma. They also function in a long-term prevention of symptoms in severe persistent asthma to suppress and control and actually reverse inflammation. The side effects associated with systemic corticosteroids are even greater than those associated with inhaled corticosteroids. Some side effects associated with longer term use include adrenal axis suppression, growth suppression, dermal thinning, hypertension, diabetes, Cushing's syndrome, cataracts, muscle weakness, and in rare instances, impaired immune function.

The combination of the compounds of the invention and steroids are particularly well suited to the treatment of young subjects (e.g., children). To date, the use of steroids in children has been limited by the observation that some steroid treatments have been reportedly associated with growth retardation. Thus, according to the present invention, the compounds of the invention can be used in combination with steroids, allowing for the use of lower required doses of steroids.

Corticosteroids include, but are not limited to, beclomethasone dipropionate (inhaler: Becotive™/Qvar™; nasal spray: Beconase™/Vancenase™), budesonide (Rhinocort™/Pulmicort™, AstraZeneca), flunisolide (AeroBid™/Nasaline™/Nasarel™), fluticasone propionate (Flovent™/Flonase™, GlaxoSmithKline; Flixotide™/Flixonase™, Allen & Hanburys), fluticasone furoate (Veramyst™, GlaxoSmithKline) and triamcinolone (Kenalog™/Aristocort™/Nasacort™/Tri-Nasal™/Triderm™/Azmacort™/Trilone™/Volon A™/Tristoject™/Fougera™/Tricortone™/Triesence™). Although dexamethasone is a corticosteroid having anti-inflammatory action, it is not regularly used for the treatment of asthma/allergy in an inhaled form because it is highly absorbed, it has long-term suppressive side effects at an effective dose. Dexamethasone, can be used according to the invention for the treating of asthma/allergy because, when administered in combination with the compounds of the the invention, it can be administered at a low dose thereby reducing the side effects. Additionally, the compounds of the invention can be administered to reduce the side effects of dexamethasone even at higher concentrations. Some of the side effects associated with corticosteroid include cough, dysphonia, oral thrush (candidiasis), and in higher doses, systemic effects, such as adrenal suppression, osteoporosis, growth suppression, skin thinning and easy bruising.

Systemic corticosteroids include, but are not limited to, methylprednisolone (Medrol™/Solu-Medrol™, Sandoz), prednisolone (Teva, KV Pharmaceutical) and prednisone (Deltasoneprednisone™, Pharmacia & UpJohn).

Inhaled glucocorticoids are the most widely used prevention medications and normally come as inhaler devices: ciclesonide (Alvesco™, Nycomed), beclomethasone (inhaler: Becotive™/Qvar™; nasal spray: Beconase™/Vancenase™), budesonide (Rhinocort™/Pulmicort™, AstraZeneca), flunisolide (AeroBid™/Nasaline™/Nasarel™), fluticasone (Flovent™/Flonase™/Veramyst™, GlaxoSmithKline; Flixotide™/Flixonase™, Allen & Hanburys), mometasone (Nasonex™/Asmanex Twisthaler™, Schering-Plough), and triamcinolone (Kenalog™/Aristocort™/Nasacort™/Tri-Nasal™/Triderm™/Azmacort™/Trilone™/Volon A™/Tristoject™/Fougera™/Tricortone™/Triesence™). Due to the deleterious side effects of use of corticosteroids, inhaled steroids are generally used for prevention, as their smaller doses are targeted to the lungs, unlike the higher doses of oral preparations. Nevertheless, patients on high doses of inhaled steroids may still require prophylactic treatment to prevent osteoporosis. Deposition of steroids in the mouth may cause a hoarse voice or oral thrush (due to decreased immunity). This may be minimized by rinsing the mouth with water after inhaler use, as well as by using a spacer which increases the amount of drug that reaches the lungs.

Immunomodulators include, but are not limited to, anti-inflammatory agents, leukotriene antagonists, IL-4 muteins, soluble IL-4 receptors, immunosuppressants, anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, downregulators of IgE, and the like.

Leukotriene modifiers are often used for long-term control and prevention of symptoms in mild persistent asthma. Leukotrienes are biochemical mediators that are released from mast cells, eosinophils, and basophils that cause contraction of airway smooth muscle and increase vascular permeability, mucous secretions and activate inflammatory cells in the airways of patients with asthma. Leukotriene modifiers function as leukotriene receptor antagonists by selectively competing for LTD-4 and LTE-4 receptors. These compounds include, but are not limited to, montelukast (Singulair™, Merck), zafirlukast (Accolate™/Accoleit™/Vanticon™, AstraZeneca), pranlukast and zileuton (Zyflo™, Abbott). Zileuton tablets function as 5-lipoxygenase inhibitors.

Down-regulators of IgE include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. One of skill in the art can prepare functionally active antibody fragments of binding peptides with the same function. Other types of IgE downregulators are polypeptides capable of blocking the binding of the IgE antibody to the Fc receptors on the cell surfaces and displacing IgE from binding sites upon which IgE is already bound. An example of an IgE blocker is omalizumab (Xolair™), a recombinant DNA-derived IgGlk monoclonal antibody that binds selectively to IgE and is made by Genentech/Novartis.

Mast cell stabilizers, as the name implies, stabilize mast cell membranes and inhibit activation and release of mediators from eosinophils and epithelial cells. Such compounds, exemplified by cromolyn sodium (cromoglicic acid; nasal spray: Rynacrom™ (UK), Nasalcrom™, Prevalin™ (Netherlands); inhaler: Intal™; oral form: Gastrocrom™) and nedocromil (inhaler: Tilade™; eye drop: Alocril™), are used as long-term control medications for preventing primarily asthma symptoms arising from exercise or allergic symptoms arising from allergens. These compounds are believed to block early and late reactions to allergens by interfering with chloride channel function.

An anticholinergic agent is a substance that blocks the neurotransmitter acetylcholine in the central and the peripheral nervous system. Frequently, they reduce the effects mediated by acetylcholine on acetylcholine receptors in neurons through competitive inhibition. Therefore, their effects are reversible. Anticholinergics are classified according to the receptors that are affected: (a) antimuscarinic agents operate on the muscarinic acetylcholine receptors; the majority of anticholinergic drugs are antimuscarinics; and (b) antinicotinic agents operate on the nicotinic acetylcholine receptors.

Anticholinergics are generally used for the relief of acute bronchospasm. These compounds are believed to function by competitive inhibition of muscarinic cholinergic receptors. Anticholinergics include, but are not limited to, ipratrapoium bromide (Atrovent™/Apovent™, Boehringer Ingelheim), oxitropium and tiotropium (Spiriva™, Boehringer-Ingelheim/Pfizer). These compounds reverse only cholinerigically-mediated bronchospasm and do not modify any reaction to antigen. Side effects include drying of the mouth and respiratory secretions, increased wheezing in some individuals, blurred vision if sprayed in the eyes. Ipratropium is also combined with albuterol (trade names Combivent™ and Duoneb™) for the management of chronic obstructive pulmonary disease (COPD) and asthma, and with fenoterol (trade names Duovent™ and Berodual N™) for the management of asthma.

Methotrexate is an antimetabolite and antifolate drug used in treatment of cancer and autoimmune diseases. It acts by inhibiting the metabolism of folic acid. It has come into use as a treatment for some autoimmune diseases, including ankylosing spondylitis, Crohn's disease, psoriasis, psoriatic arthritis, rheumatoid arthritis, and scleroderma, along with difficult-to-treat asthma cases.

Another method currently used for treating allergic disease involves the injection of increasing doses of allergen to induce tolerance to the allergen and to prevent further allergic reactions. Allergen injection therapy (allergen immunotherapy) is known to reduce the severity of allergic rhinitis. Other attempts to treat allergy involve modifying the allergen chemically so that its ability to cause an immune response in the patient is unchanged, while its ability to cause an allergic reaction is substantially altered. These methods, however, are associated with the risk of side effects such as anaphylactic shock. Therefore, the use of a composition of the invention and an asthma/allergy medicament in combination with an allergen can avoid many of the side effects.

In some cases the subject is exposed to an allergen in addition to being treated with a composition of the invention either in the absence or presence of other asthma/allergy medicament. In this case the subject is said to be exposed to the allergen. As used herein, the term “exposed to” refers to either the active step of contacting the subject with an allergen or the passive exposure of the subject to the allergen in vivo. Methods of the active exposure of a subject to an allergen are well-known in the art. In general, an allergen is administered directly to the subject by any means such as intravenous, intramuscular, oral, transdermal, mucosal, intranasal, intratracheal, or subcutaneous administration. The allergen can be administered systemically or locally. A subject is passively exposed to an allergen if an allergen becomes available for exposure to the immune cells in the body. A subject may be passively exposed to an allergen, for instance, by entry of an allergen into the body when the allergen is present in the environment surrounding the subject, i.e. pollen.

As used herein, the term “prevent”, “prevented”, or “preventing” when used with respect to the treatment of an allergic or asthmatic disorder refers to a prophylactic treatment which increases the resistance of a subject to an allergen, in other words, decreases the likelihood that the subject will develop an allergic or asthmatic response to the allergen as well as a treatment after the allergic or asthmatic disorder has begun in order to fight the allergy/asthma, e.g., reduce or eliminate it altogether or prevent it from becoming worse.

In another aspect, the invention includes a method of decreasing the dose of an asthma/allergy medicament by administering to a subject having asthma or allergy or at risk of developing asthma or allergy an asthma/allergy medicament in a sub-therapeutic dosage in combination with a compound of the invention, wherein the combination of the sub-therapeutic dose of the asthma/allergy medicament and the compound of the invention produce a therapeutic result in the prevention or treatment of asthma or allergy in the subject. The method allows a lower dose of the asthma/allergy medicament to be used. This provides several advantages, including lower costs associated with using less drugs and less chances of inducing side effects resulting from the medications by using lower doses.

In other aspects, the invention is a method of altering the dosage of the asthma/allergy medicament that is required to treat a subject suffering from asthma or allergy. The invention in one aspect is a method of increasing the dose of an asthma/allergy medicament without inducing the level of side effects ordinarily observed with that dose of an asthma/allergy medicament. The method is accomplished by administering to a subject suffering from asthma or allergy or at risk of developing asthma or allergy, an asthma/allergy medicament in a dose which would ordinarily induce side effects in combination with a compound pound of the invention to the subject, wherein administration of the compound of the invention prevents the side effects associated with the high dose of the asthma/allergy medicament. The method provides a basis for administering higher therapeutic doses of an asthma/allergy medicament to a subject in order to prevent or reduce the symptoms associated with an asthmatic or an allergic response more sufficiently than a lower dose. It is not desirable to administer such high doses alone, in the absence of a compound of the invention because of the side effects resulting from the high dose of the asthma/allergy medicament.

According to other aspects, the invention provides methods of treating or preventing asthma and/or allergy by administering a compound of the invention and an asthma/allergy medicament in different dosing schedules. In one aspect, the invention is a method of preventing or treating asthma or allergy by administering to a subject a compound of the invention in an effective amount for modulating the immune response and subsequently administering to the subject an asthma/allergy medicament. In other aspects, the invention is a method of preventing or treating asthma or allergy by administering to a subject an allergy/asthma medicament in an effective amount for providing some symptomatic relief and subsequently administering a compound of the invention to the subject.

The compounds of the invention, alone or in combinations with other therapeutic agents described herein can be administered to a cell, a tissue, or an animal to decrease lung resistance in a diseased lung. Methods of the safe and effective administration of the compounds of the invention are known to those skilled in the art. For instance, the administration of fluphenazine is described in the standard literature. That is, the administration of many serotonin-affecting agents, compounds of the invention, and fluphenazine is set forth in the Physician's Desk Reference (1996 edition, Medical Economics Co., Montvale, N.J.), the disclosure of which is incorporated by reference as if set forth in its entirety herein.

Subject doses of the compounds of the invention typically range from about 0.1 μg/day to 10,000 mg/day, more typically from about 1 μg/day to 1000 mg/day, and most typically from about 10 μg/day to 100 mg/day and any and all whole or partial increments there between.

Stated in terms of subject body weight, typical dosages range from about 0.1 μg/kg/day to 1000 mg/kg/day, more typically from about 10 μg/kg/day to 500 mg/kg/day, more typically from about 20 μg/kg/day to 100 mg/kg/day, more typically from about 50 μg/kg/day to 50 mg/kg/day, and most typically from about 0.10 mg/kg/day to 5 mg/kg/day and any and all whole or partial increments there between.

Subject oral doses of the compounds of the invention typically range from about 0.1 μg/day to 10,000 mg/day, more typically from about 1 μg/day to 1000 mg/day, yet more typically from about 10 μg/day to 100 mg/day, and most typically 8 mg/day to 80 mg/day and any and all whole or partial increments there between.

Stated in terms of subject body weight, typical oral dosages range from about 0.1 μg/kg/day to 1000 mg/kg/day, more typically from about 10 μg/kg/day to 500 mg/kg/day, more typically from about 20 μg/kg/day to 100 mg/kg/day, more typically from about 50 μg/kg/day to 50 mg/kg/day, and most typically from about 0.10 mg/kg/day to 5 mg/kg/day and any and all whole or partial increments there between.

The compositions of the invention for administration can be administered in,a dose range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, and any and all whole or partial increments there between.

In some embodiments, the dose of a composition of the invention is between about 0.0001 mg and about 25 mg. In some embodiments, a dose of a composition of the invention used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound (i.e., asthma/allergy medicament) as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments there between.

### III. Pharmaceutical Composition

For administration of a compound of the present invention to a mammal, the compound can be suspended in any pharmaceutically acceptable carrier, for example, sterile water or a buffered aqueous carriers, such as glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

The compounds of the invention are preferably administered to the subject as a pharmaceutical or veterinary composition, which includes systemic and topical formulations. Among these, preferred are formulations suitable for inhalation, or for respirable, buccal, oral, rectal, vaginal, nasal, intrapulmonary, ophthalmic, optical, intracavitary, intratraccheal, intraorgan, topical (including buccal, sublingual, dermal and intraocular), parenteral (including subcutaneous, intradermal, intramuscular, intravenous and intraarticular) and transdermal administration, among others. The route(s) of administration will be readily apparent to the skilled artisan and will depend upon any number of factors including the type and severity of the disease being treated, the type and age of the veterinary or human patient being treated.

The compounds of the invention may be administered to the lungs of a subject by any suitable means, but are preferably administered by generating an aerosol or spray comprised of respirable, inhalable, nasal or intrapulmonarily delivered particles comprising the active compound, which particles the subject inhales, i.e. by inhalation administration. The respirable particles may be liquid or solid. Particles comprising the active compound for practicing the present invention should include particles of respirable or inhalable size; that is, particles of a size sufficiently small to pass through the mouth and larynx upon inhalation and into the bronchi and alveoli of the lungs. In general, particles ranging from about 0.05, about 0.1, about 0.5, about 1, about 1.5 to about 5, about 6, about 7, about 8, about 10 microns in size, more particularly particles about 0.5 to less than about 5 microns in size, are respirable or inhalable. When particles of nonrespirable size are included in the aerosol or spray, they tend to deposit in the throat and be swallowed. Thus, the quantity of non-respirable particles in the aerosol or spray is preferably minimized when intended for respirable administration or by inhalation. For nasal or intrapulmonary administration, a particle size in the range of about 10, about 11, about 15, about 20 to about 25, about 30, about 40, about 50, and sometimes even up to about 100 and about 500 microns is preferred to ensure retention in the nasal or pulmonary cavity. Pulmonary instillation is particularly useful for treating newborns.

Liquid pharmaceutical compositions of the active compound for producing an aerosol or spray may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optional contain a dispersant which serves to facilitate the formation of an aerosol. A suitable dispersant is lactose, which may be blended with the active compound in any suitable ratio, e.g. a 1 to 1 ratio by weight. Other therapeutic and formulation compounds may also be included, such as a surfactant to improve the sate of surfactant in the lung and help with the absorption of the active agent.

Aerosols of liquid particles comprising the active compound may be produced by any suitable means, such as with a nebulizer. See, e.g., U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w of the compositions, but preferably less than 20% w/w he carrier is typically water or a dilute aqueous alcoholic solution, preferably made isotonic with body fluids by the addition of, for example sodium chloride. Optional additives include preservatives if the compositions is not prepared sterile, for example, methyl hydroxybenzoate, antioxidants, flavoring agents, volatile oils, buffering agents and surfactants.

Aerosols of solid particles comprising the active compound may likewise be produced with any sold particulate medicament aerosol generator. Aerosol generators for administering solid particulate medicaments to a subject product particles which are respirable, as explained above, and they generate a volume of aerosol containing a predetermined metered dose of a medicament at a rate suitable for human administration. Examples of such aerosol generators include metered dose inhalers and insufflators.

Pharmaceutical compositions that are useful in the methods of the invention may be administered systemically in oral solid formulations, ophthalmic, suppository, aerosol, topical or other similar formulations. In addition to the compounds of the invention, or a biological equivalent thereof, such pharmaceutical compositions may contain pharmaceutically-acceptable carriers and other ingredients known to enhance and facilitate drug administration.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

In yet another embodiment, compositions of the invention may be administered to the desired location of a mammal by a transdermal patch. A transdermal patch is meant a system capable of delivery of a compound to a mammal via the skin, or any suitable external surface, including mucosal membranes, such as those found inside the mouth. Such delivery systems generally comprise a flexible backing, an adhesive and a compound retaining matrix, the backing protecting the adhesive and matrix and the adhesive holding the whole on the skin of the mammal. On contact with the skin, the compound-retaining matrix delivers the compound to the skin, the compound then passing through the skin into the mammal's system.

Certain embodiments of the invention provide a pharmaceutical preparation/dosage formulation provided in the form of a transdermal patch and formulated for sustained release formulation, in a therapeutically effective amount sufficient to treat a disease associated with activation of an immune cell (e.g., rheumatoid arthritis) in a patient, wherein the dosage formulation, when administered (provided as a patch) to the patient, provides a substantially sustained dose over at least about 2 hours, 4 hours, 6 hours, 8, hours, 12 hours, 20 hours, or at least about 24 hours.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

A pharmaceutical composition of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like.

### IV. Kits

The present invention also provides a kit comprising a composition of the invention and a delivery device. The compositions may conveniently be presented in single or multiple unit dosage forms as well as in bulk, and may be prepared by any of the methods which are well known in the art of pharmacy. The composition, found in the kit, whether already formulated together or where the compounds are separately provided along with other ingredients, and instructions for its formulation and administration regime. The kit may also contain other agents, such as those described elsewhere herein and, for example, when for parenteral administration, they may be provided with a carrier in a separate container, where the carrier may be sterile. The present composition may also be provided in lyophilized form, and in a separate container, which may be sterile, for addition of a liquid carrier prior to administration.

Additionally, one skilled in the art would appreciate, based upon the disclosure provided herein, that the compound of the invention can be a compound that does not cross the blood-brain barrier and is preferably water soluble. This is because, as more fully discussed elsewhere herein, it may be desirable to inhibit serotonin signaling in a non-neural cell, while not affecting such signaling in a neural cell, which would be protected beyond the blood-brain barrier.

In a specific embodiment, the kit of the present invention comprises a compound of the invention, an applicator, and an instructional material for the use thereof. In another embodiment, the kit can comprise a compound of formula I, such as those described elsewhere herein, a container holding the compound, and an instructional material. The skilled artisan can provide the applicator.

Preferably, the kit of the present invention comprises a compound of formula I, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, or ICI-1505. More preferably, the kit comprises ICI-735. Additionally, the kit can comprise an instructional material and an applicator for the administration of the compounds of the present invention for the treatment of a respiratory disease or condition. The kits of the present invention can be used to treat the diseases and conditions disclosed elsewhere herein. The kits described in the present invention are not limited to the uses above however, and can be used in any method derived from the teachings disclosed herein.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Examples

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 1

### Selection of Candidate Agents

A criterion for selecting an agent for proceeding to clinical trials is whether the compound crosses the blood-brain barrier. In the experiments discussed herein, it is desirable to select a compound that does not cross the blood-brain barrier. Fluphenazine (ICI-01175) is used as a control compound that has the potential to cross the blood-brain barrier. Table 1 summarizes the results of the test compounds for their potential to cross the blood-brain barrier. All tested compounds were observed to have a low potential to cross the blood-brain barrier.

### TABLE 1: Potential to Cross the Blood-Brain Barrier

MPR1-MDCK Permeability

| | | | | | | Brain |
|---|---|---|---|---|---|---|
| Test | % Recovery | | Papp (×10−6 cm/s) | | | Penetration |
| Compound ID # | A-B | B-A | A-B | B-A | Efflux | Classification |
| ICI-00847 | 9 | 31 | 0.56 | 9.56 | 17 | Low |
| ICI-00890 | 15 | 29 | 0.75 | 8.60 | 11 | Low |
| ICI-00894 | 56 | 56 | 0.22 | 30.0 | 136 | Low |
| ICI-00715 | 85 | 77 | 0.08 | 12.3 | 154 | Low |
| ICI-00685 | 17 | 15 | 0.66 | 4.47 | 6.8 | Low |
| ICI-00850 | 11 | 9 | 0.94 | 4.94 | 5.3 | Low |
| ICI-00954 | 69 | 18 | 0.15 | 19.6 | 132 | Low |
| ICI-00953 | 23 | 7 | 0.47 | 5.31 | 11 | Low |
| ICI-00848 | 22 | 21 | 0.24 | 15.5 | 64 | Low |
| ICI-01505 | 3 | 26 | <0.36 | 0.64 | >1.8 | Low |
| ICI-00758 | 59 | 41 | 0.21 | 24.9 | 116 | Low |
| ICI-00735 | 47 | 33 | 0.23 | 28.6 | 124 | Low |
| ICI-01417 | 75 | 79 | 2.05 | 87.7 | 43 | Low |
| ICI-01260 | 55 | 47 | 0.32 | 26.4 | 82 | Low |
| ICI-01175 | 24 | 43 | 8.70 | 9.63 | 1.1 | High |
| ICI-01176 | 35 | 55 | 0.78 | 34.2 | 44 | Low |
| ICI-01008 | 3 | 1 | 0.20 | 0.21 | 1.0 | Low |
| ICI-01259 | 29 | 39 | <0.29 | 12.5 | >43 | Low |
| ICI-00760 | 46 | 40 | <0.25 | 18.8 | >75 | Low |
| ICI-00761 | | | | | | |
| ICI-00824 | 11 | 21 | 1.77 | 13.8 | 7.8 | Low |
| ICI-00846 | 3 | 1 | <0.36 | <0.49 | ND | Low |
| ICI-00849 | 11 | 26 | <1.00 | 5.10 | >5.1 | Low |
| ICI-01007 | 19 | 36 | 0.79 | 16.7 | 21 | Low |
| ICI-00822 | 61 | 22 | 0.13 | 15.7 | 119 | Low |
| ICI-00823 | 57 | 46 | <0.24 | 19.4 | >80 | Low |
| ICI-00738 | 15 | 40 | 1.06 | 3.33 | 3.1 | Low |

Brain Penetrating Potential Classification:
1) A-B Papp > 3.0 and Efflux Ratio < 3.0: High
2) A-B Papp > 3.0 and 10 > Efflux Ratio > 3.0: Moderate
3) A-B Papp > 3.0 and Efflux Ratio > 10: Low
4) A-B Papp < 3.0: Low

The results presented herein demonstrate that adding various side groups to a compound such as Fluphenazine, decreases the ability of the modified Fluphenazine to cross the blood-brain barrier. Without wishing to be bound by any particular theory, modification of fluphenazine can reduce the ability of the compound to cross the blood-brain barrier.

The next set of experiments was designed to assess compounds that have low levels of brain penetration for the ability of the test compounds to inhibit cellular proliferation. An exemplary in vitro assay is to screen for the ability of the test compounds to inhibit proliferation of the B-cell neoplastic cell line RPMI 8226 (a plasmacytoma derived from a multiple myeloma patient (Matsuoka, et al., 1967, Proc. Soc. Exp. Biol. Med. 125: 1246-1250). However other cell line can be used such as, U266 (established from an IgE-secreting myeloma patient (Nilsson, et al., 1970, Clin. Exp. Immunol., 7: 477-489) and ARH77 (an EBV transformed plasma cell leukemia (Burk, et al., 1978, Cancer Res. 38: 2508-2513).

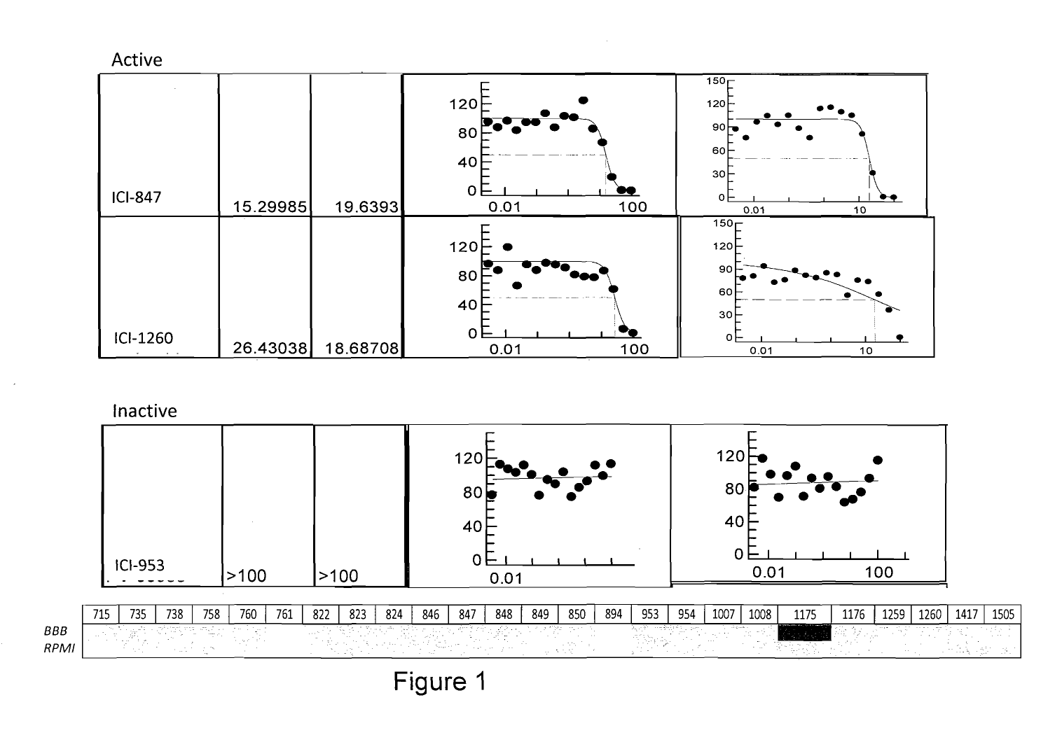

It was observed that among the compounds tested, ICI-715, ICI-761, ICI-847, ICI-848, ICI-849, ICI-1259, and ICI-1260 was able to inhibit the proliferation of RPMI cells. An exemplary depiction of compounds that are active or otherwise able to inhibit proliferation of RPMI cells and inactive compounds (e.g., ICI-953) or otherwise not able to inhibit proliferation of RPMI cells is shown in FIG. 1. FIG. 1 is a graph depicting the effects on the test compounds on RPMI cell viability.

### Example 2

### Treatment of Asthma Using Compounds of the Invention

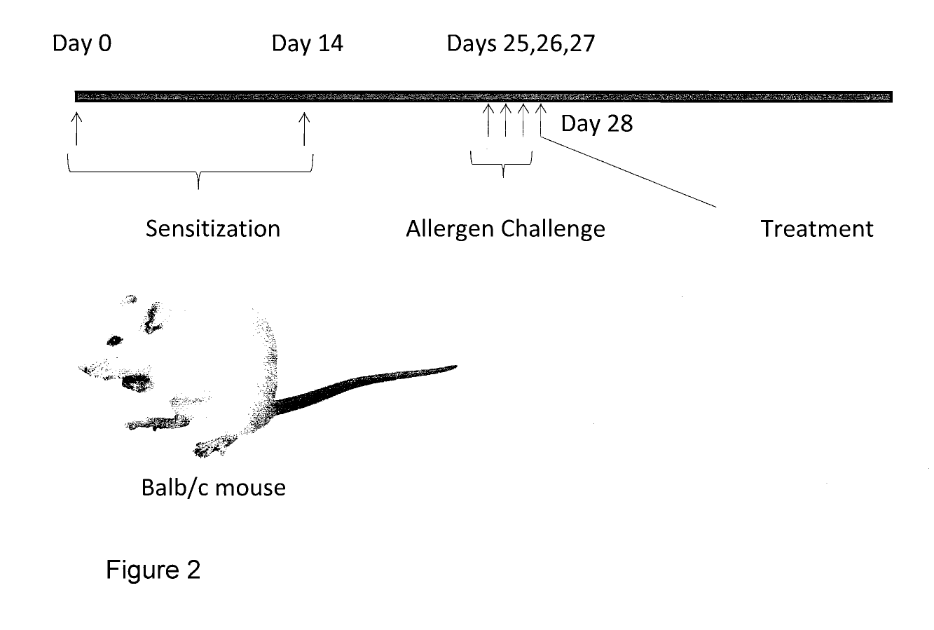

The following experiments were designed to test the efficacy of the compounds of the invention on a murine asthma model. A murine model of Aspergillus fumigatus (Af)-induced allergic airway inflammation and hyperresponsiveness was adapted from Haczku et al. (2001 Am. J. Respir. Cell Mol. Biol. 25: 45-50) to determine the activity of the candicate compounds (FIG. 2).

Briefly, BALB/c mice were sensitized intraperitoneally and challenged intranasally with Af extract as a form of a type of allergen challenge in the presence or absence of dexamethasone. With respect to sensitization, “naïve” mice received intranasal vehicle challenges only, with phosphate-buffered saline (PBS) where as “sensitized’ mice were injected intraperitoneally with 25 μg Af together with alum in 100 μl of PBS on days 1 and 14, followed by intranasal challenge on days 25, 26, and 27 with 25 μg of Af extract in 40 PBS μl in the presence or absence of dexamethasone (2.5 mg/kg in 100 μl saline). On day 28, sensitized mice were treated with the test compound (0.1, 1.0, 10 mg/kg) either one hour before or simultaneously with methacholine challenge (2.5, 5, 10, 20 mg/mL). Methacholine challenge is to induce airway constriction.

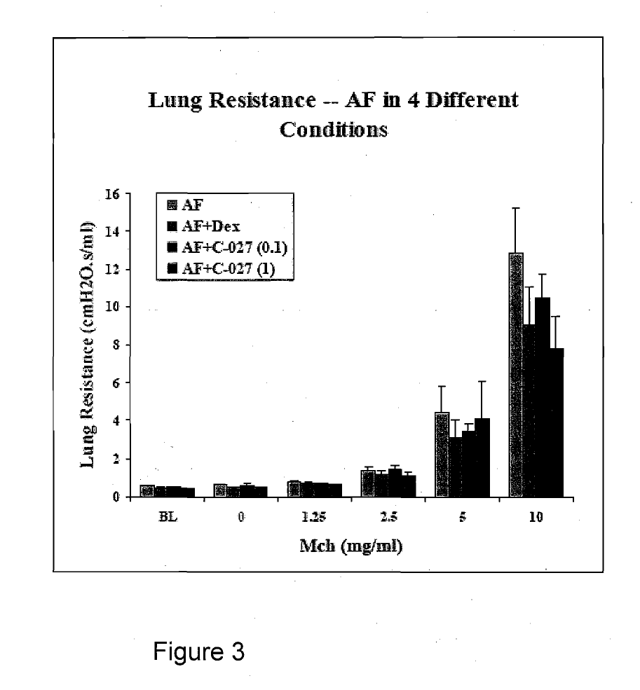

The results demonstrate that ICI-735, delivered intraperitoneally at a dose of 0.1 mg/kg, was at least as effective as 2.5 mg/kg dexamethasone at decreasing lung resistance in an Af-based mouse asthma model. In some instances, ICI-735 (C-027) was more effective than dexamethasone at decreasing lung resistance as measured by cm H₂O/ml/sec (FIG. 3). Dexamethasone is a well-known compound used for treating asthma, among other inflammatory diseases. The present experimental results therefore suggest that compounds of the invention can be useful for treating asthma and related conditions.

### Example 3

### Bronchoalveolar Lavage (BAL) Analysis for Cell Count

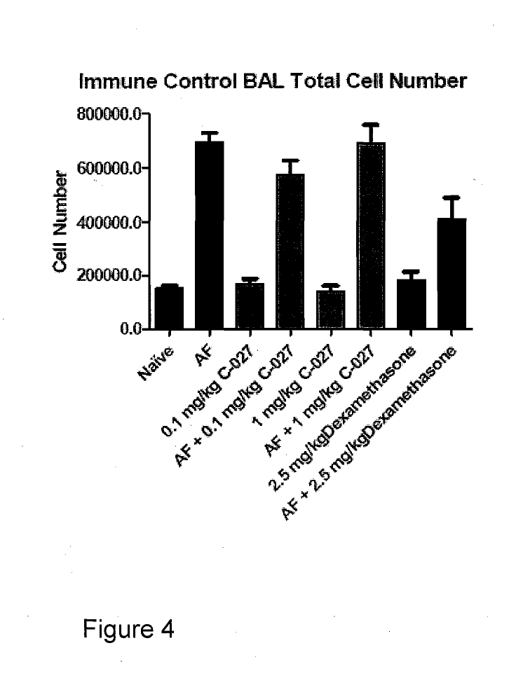

Lungs from the tested animals in Example 2 were lavaged with either a small volume (1 ml) of sterile PBS or a large volume (5 ml) of sterile saline, and total and differential cell counts were performed as described previously (Haczku et al., 1999 Am. J. Respir. Crit. Care Med. 159: 1638-1643; Haczku et al., 2000 Am. J. Respir. Crit. Care Med. 161: 952-960). There was not a significant difference in the total number of cells between asthmatic animals treated and untreated with ICI-735 (FIG. 4).

### Example 4

### Serotonin-Receptor Binding Assays

The methods employed in this study have been adapted from the scientific literature to maximize reliability and reproducibility. The experiments include contacting a serotonin type 1A, 1B, 1D, 2A, 2B, 2C, 3, 4, 5A, 6, and 7 receptor with a test compound and comparing the level of binding of serotonin or a control compound (e.g., fluphenazine) with that serotonin type 1A, 1B, 1D, 2A, 2B, 2C, 3, 4, 5A, 6, and 7 receptor with the level of serotonin binding or control compound binding with an otherwise identical serotonin type 1A, 1B, 1D, 2A, 2B, 2C, 3, 4, 5A, 6, and 7 receptor not contacted with the test compound. The skilled artisan would understand that a lower level of serotonin binding or control compound binding with the receptor contacted with the compound compared with the level of serotonin binding or control compound binding with the otherwise identical serotonin type 1A, 1B, 1D, 2A, 2B, 2C, 3, 4, 5A, 6, and 7 receptor not contacted with the compound is an indication that the compound inhibits the serotonin/receptor or control compound/receptor interaction and is, therefore, useful for treating a disease associated with the immune system. The skilled artisan would also appreciate, in view of the disclosure provided herein, that standard binding assays known in the art, or those to be developed in the future, can be used to assess the binding of serotonin or a control compound with a serotonin type 1A, 1B, 1D, 2A, 2B, 2C, 3, 4, 5A, 6, and 7 receptor in the presence or absence of the test compound to identify a useful compound.

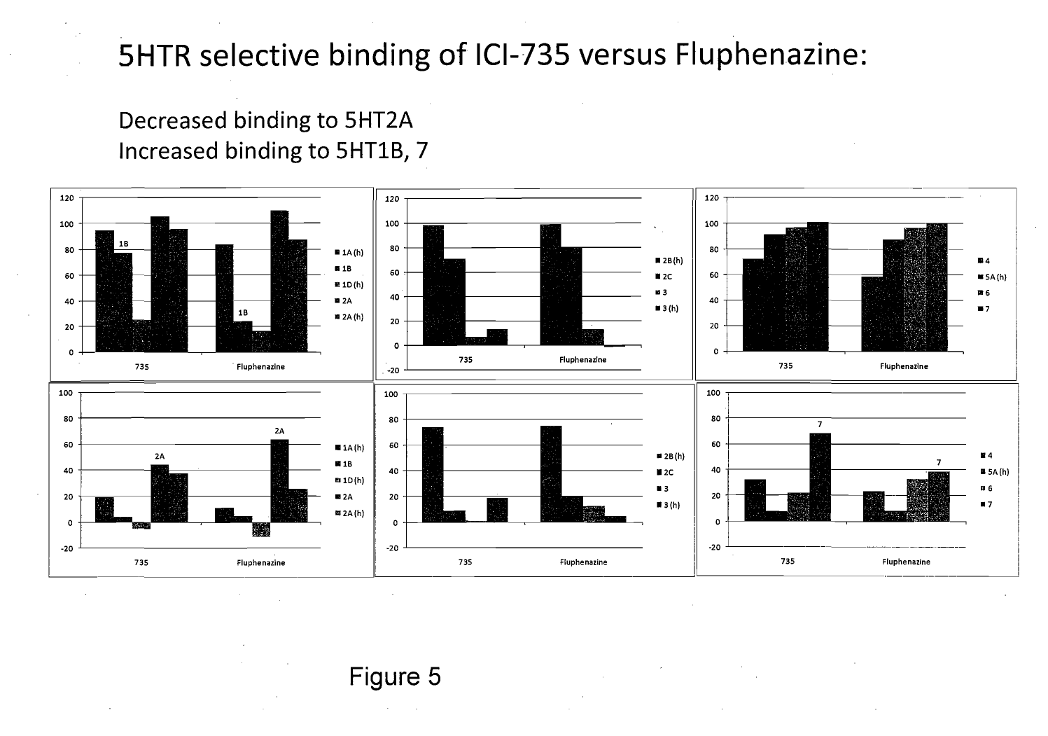

The results from the binding experiments are summarized in FIG. 5. ICI-735 and fluphenazine were tested for selective binding to 1A, 1B, 1D, 2A, 2B, 2C, 3, 4, 5A, 6, and 7 receptors at concentrations of 10 μM and 0.1 μM. The 5HTR binding data presented herein suggest a significant increase activity for 1B and 7 receptor and a decrease activity for 2A receptor with respect to fluphenazine.

### Example 5

### Pharmacokinetics of ICI-735

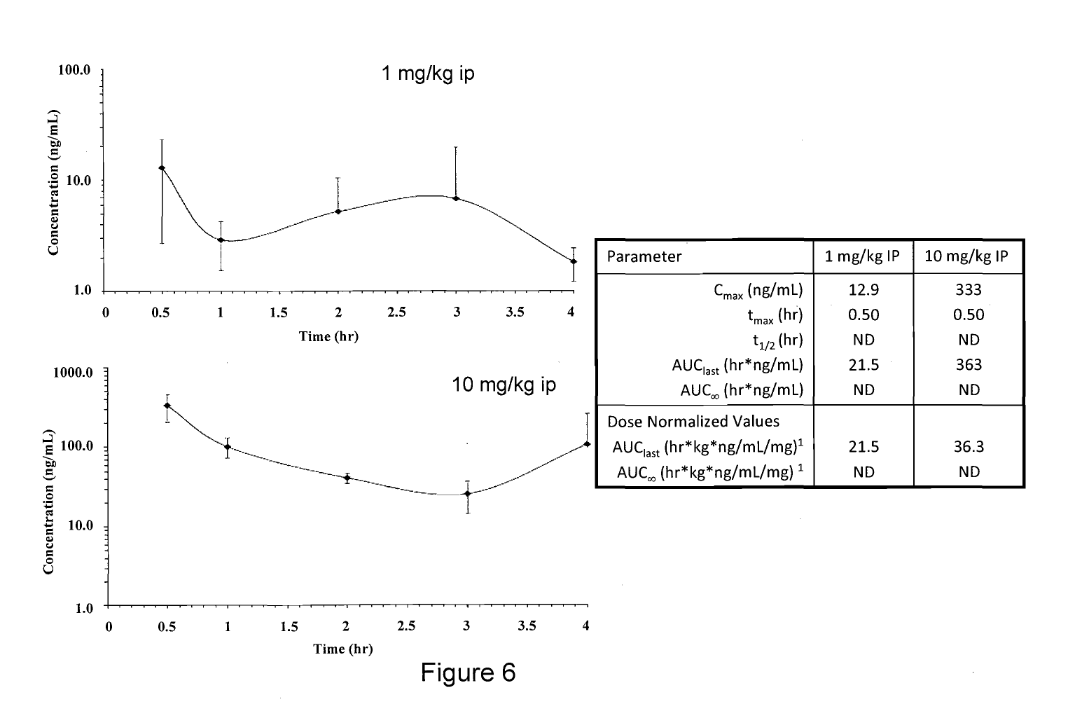

Mice were treated with either 1.0 mg/kg or 10 mg/kg of ICI-735 by way of intraperitoneal administration. The plasma levels of ICI-735 were measured at baseline and various time points following administration (FIG. 6). It was observed that plasma levels of ICI-735 after i.p. 1.0 mg/kg dose were comparable to levels after i.p. 10 mg/kg dose. Without wishing to be bound by any particular theory, it is believed that the effectiveness of ICI-735 to treat asthma can be achieved with a low dose (e.g, 1.0 mg/kg dose).

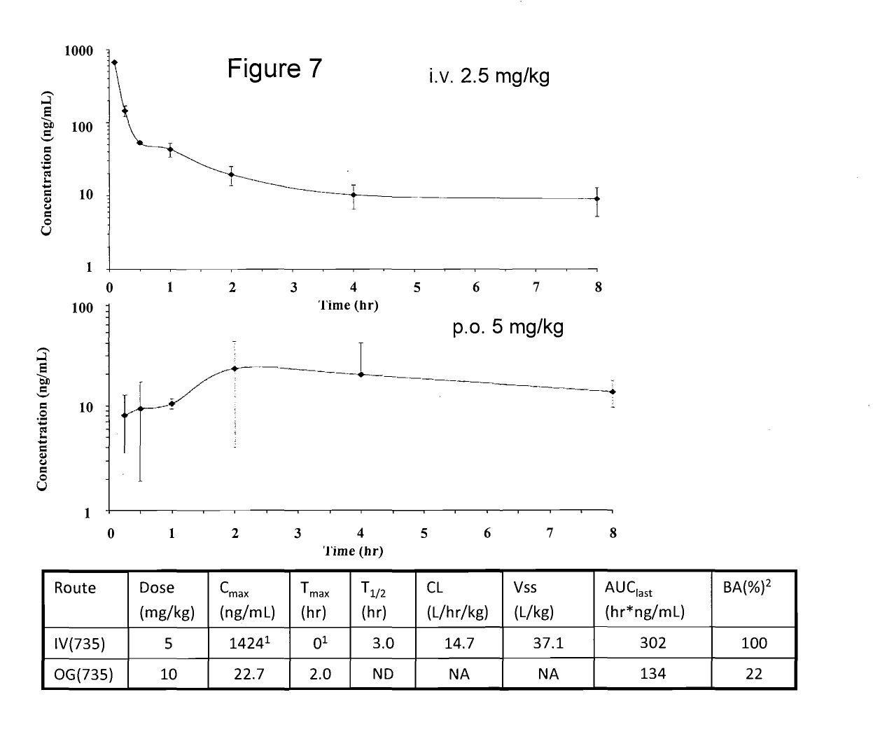

Mice were also treated with either 2.5 mg/kg intravenously or 5 mg/kg orally of ICI-735. The plasma levels of ICI-735 were measured at baseline and various time points following administration (FIG. 7). It was observed that plasma levels of ICI-735 after oral 5 mg/kg dose were comparable to levels after intravenous 2.5 mg/kg dose. It was determined that oral bioavailability was approximately 20%. Therefore, it is believed that ICI-735 administration orally at approximately 1 mg/kg/day is effective at reducing lung resistence in patients with asthma.

### Example 6

### Effects of ICI-735 on Cardiac Ion Channels and Action Potential

Voltage-gated K channels are activated by membrane depolarization. As with all voltage-gated ion channels, depolarization results in a conformational change in the S4 voltage sensor which contains positively charged amino acids. This conformational change triggers additional conformational changes leading to channel opening. Upon depolarization, these channels open, allowing the efflux of potassium from the cell, down its electrochemical gradient. They function in the heart and brain to repolarize the membrane potential and to terminate an action potential. A-type potassium currents are quickly inactivating potassium currents (eg, Kv4.2 and Kv1.4) and are important in the initial notch of repolarization in cardiac cells and the fast-spiking behavior of some neurons. Slowly inactivating channels provide more prolonged repolarization effects (eg, Kv1.5, Kv3.1, Kv2.1). hERG (human Ether-a-go-go Related Gene) channels are unusual voltage-gated potassium channels in that they are inwardly rectifying due to accelerated inactivation at positive voltages. hERG channels play a role in long-QT syndrome and block of hERG can precipitate torsades de pointe which may lead to fatal ventricular fibrillation. Screening of drugs for effects on hERG is desirable in investigational compounds.

Opening of voltage-gated calcium channels results in influx of calcium ions into the cell. This influx of calcium plays an important role in excitation-contraction coupling in cardiac and skeletal muscle fibers. The reversal potential for calcium is extremely positive, so calcium current is almost always inward, resulting in an action potential plateau in many excitable cells. These channels are the target of calcium channel blocker subtype of antihypertensive drugs. The alpha-1 subunit forms the ion conducting pore, with several modulating auxiliary subunits. Major subtypes of voltage-gated calcium channels include L-type (Cav1.1, Cav1.2, Cav1.3, Cav1.4), T-type (Cav3.1, Cav3.2, Cav3.3), N-type (Cav2.2), P-type (Cav2.1), and R-type (Cav2.3).

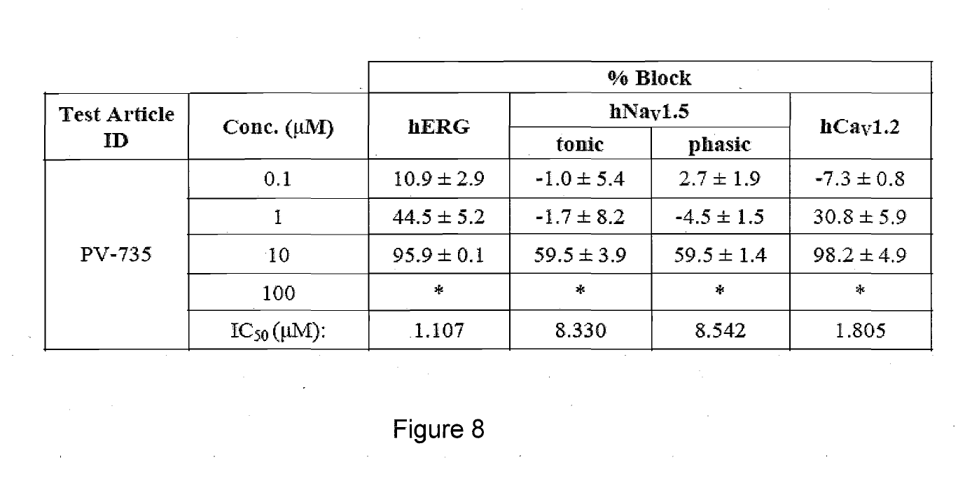

Mammalian cells were treated with ICI-735 (0.1 μM, 1 μm, 10 μm, and 100 μm) to determine whether ICI-735 blocked the expression of cardiac ion channels. It was observed that ICI-735 was able to block the expression of both hERG and hCaᵥ1.2). The results are summarized in FIG. 8. Without wishing to be bound by any particular theory, it is believed that the blocks in hERG and calcium channels may cancel one another physiologically, but sodium channel effect may lead to changes in membrane potential.

### Example 7

### ICI-735 Effects on Action Potential

Voltage-gated sodium channels are responsible for the initial phase of the action potential, which is a wave of electrical depolarisation usually initiated at the soma of the neuron and propagated along the nerve axon to the terminals. At the terminals, the action potential triggers the influx of calcium and the release of neurotransmitter. The voltage-gated sodium channel family is made up of four brain specific subtypes, Naᵥ1.1, 1.2, 1.3 and 1.6; as well as Naᵥ1.4, which is found on skeletal muscle; Naᵥ1.5, which is found on cardiac muscle; and Naᵥ1.7, 1.8, 1.9, are found predominantly on sensory neurons.

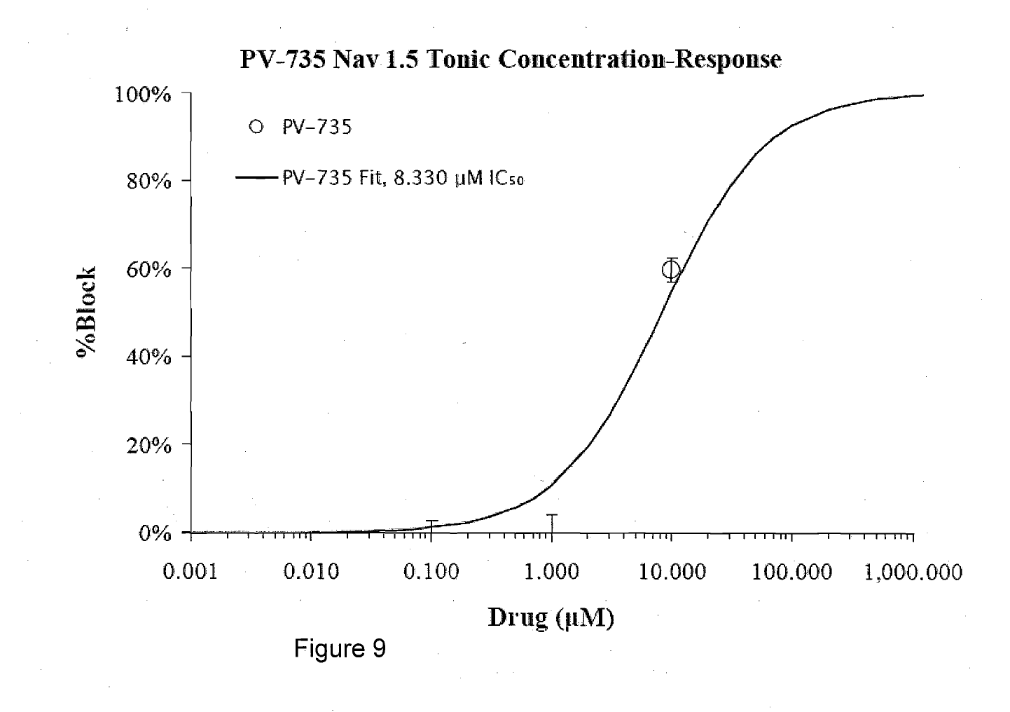

The effects of ICI-735 on action potential in a mammalian cell was tested. The results from the action potential experiment is summarized in FIG. 9. Briefly, it was observed that ICI-735 was able to block Naᵥ1.5. Specifically, the IC50 for ICI-735 was calculated to be 8.3330 μM.

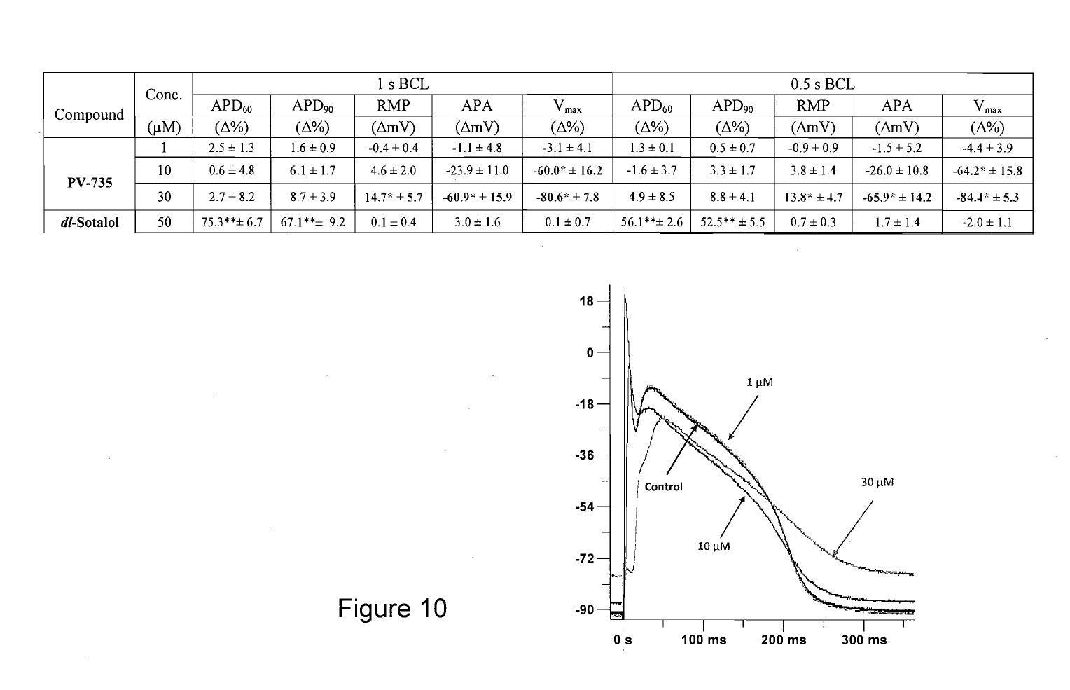

Further assessment in rabbit Punkinje fibers was undertaken to directly measure concentration-dependent changes in action potential induced by ICI-735. The results are summarized in FIG. 10. Without wishing to be bound by any particular theory, it is believed that the lack of effect on action potential duration at 60% (APD₆ₒ) and APD₉₀ is related to the parallel effects on hERG and calcium channels canceling on another.

### Example 8

### Treatment of Asthma Using Compounds of the Invention (Oral Administration)

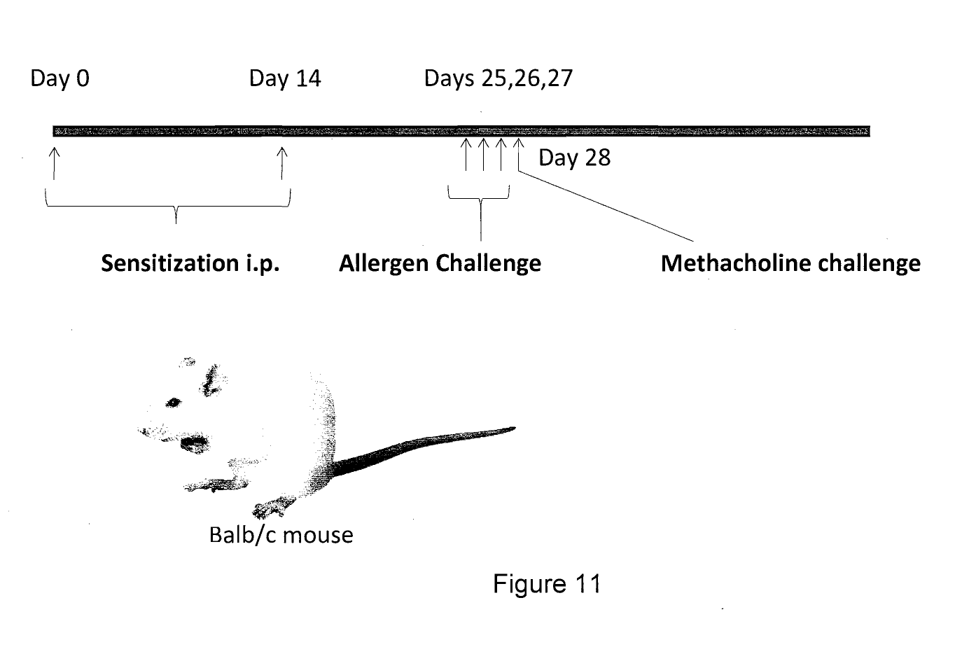

The following experiments were designed to test the efficacy of the compounds of the invention on a murine asthma model. A murine model of Aspergillus fumigatus (Af)-induced allergic airway inflammation and hyperresponsiveness was adapted from Haczku et al. (2001 Am. J. Respir. Cell Mol. Biol. 25: 45-50) to determine the activity of the eradicate compounds (FIG. 11).

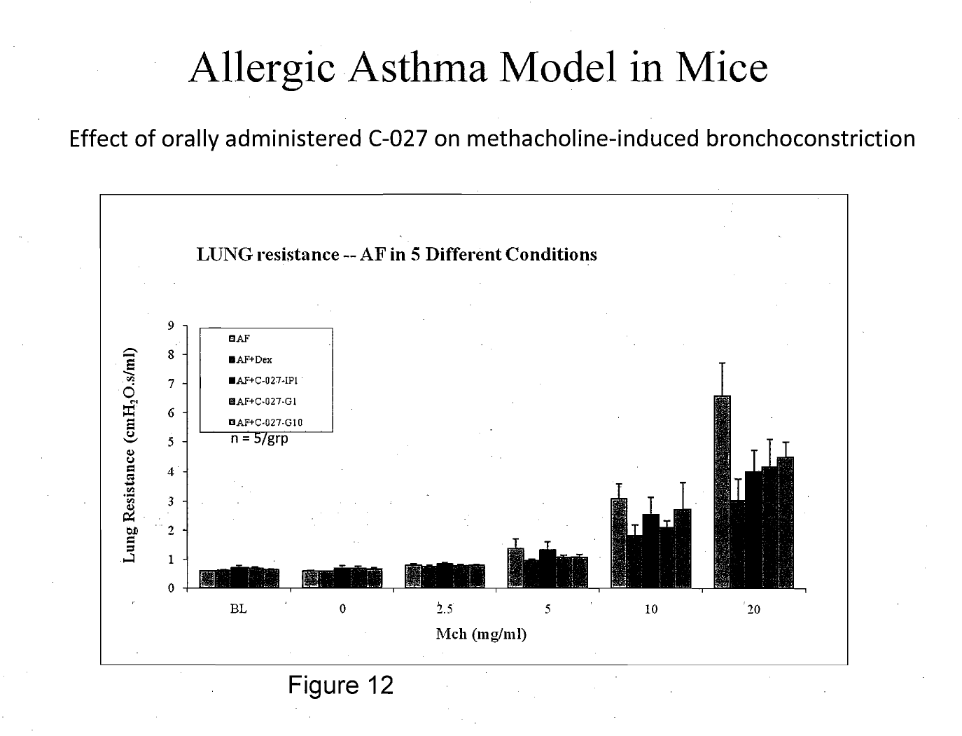

Briefly, BALB/c mice were sensitized intraperitoneally and challenged intranasally with Af extract as a form of a type of allergen challenge in the presence or absence of dexamethasone. With respect to sensitization, “naïve” mice received intranasal vehicle challenges only, with phosphate-buffered saline (PBS) where as “sensitized’ mice were injected intraperitoneally with 25 μg Af together with alum in 100 μl of PBS on days 1 and 14, followed by intranasal challenge on days 25, 26, and 27 with 25 μg of Af extract in 40 PBS μl in the presence or absence of dexamethasone (2.5 mg/kg in 100 μl saline) in combination with either o.g. treatment with ICI-735 (1 mg/kg or 10 mg/kg) or i.p. treatment with ICI-735 1 mg/kg. On day 28, sensitized mice were treated with methacholine challenge (2.5, 5, 10, 20 mg/mL). Methacholine challenge is to induce airway constriction. The results demonstrate that ICI-735, delivered orally is effective at decreasing lung resistance in an Af-based mouse asthma model (FIG. 12).

With respect to the murine model of allergen induced AHR, BALB/c mice were sensitized and challenged via intranasal instillation of Aspergillus fumigatus (Af). Two hours prior to each intranasal challenge, cohorts of five mice were administered intra peritonially (i.p.) with ICI-735 (1 mg/ml), dexamethasone (10 mg/ml). Four hours after the last allergen challenge, airway function was assessed by flexivent. Differential cytokine and immune cell abundance were also accessed in BAL fluid.

It was observed that ICI-735 significantly inhibited AHR in Af sensitized mice with no effect on naive animal airway contraction. Unlike dexamethasone, ICI-735 treatment had little effect on Af-induced cell increases and cytokine production in BAL. ICI-735 was also shown to antagonize muscarinic, histamine-induced contraction and bronchoconstriction elicited by passive sensitization in human small airways.

ICI-735 at doses as low as 0.1 mg/kg i.p. or 1 mg/kg o.g., decreased airway hype r-responsiveness to Mch in the murine acute allergic asthma model to the same extent as i.p. treatment with 2.5 mg/kg of dexamethasone. Unlike dexamethasone, ICI-735 exerted no significant effects on either immune cell infiltration of the airways or levels of allergy-associated soluble cytokines, at least at the time intervals sampled in these experiments. When administered either via the parenteral or oral route, ICI-735 may exert a favorable pharmacological effect in the lung by diminishing cholinergic hyper-responsiveness in the airway, an important symptom of patients with several respiratory inflammation diseases.

The observation that ICI-735 decreased airway hyperresponsiveness in animals dosed one hour prior to Mch challenge suggests that ICI-735 may exert effects directly on airway smooth muscle. Interestingly, ICI-735 may act via a mechanism independent of currently approved bronchodilators considering that ICI-735 does not bind the AdrR2 receptor nor a panel of leukotriene and prostanoid receptors, and exhibits only weak binding and weak in vitro functional antagonism for the muscarinic receptor M3. Because sensitivity to contractile agonists, including histamine, serotonin, leukotrienes, and prostanoids, is qualitatively and quantitatively different in rodent versus human airways, human precision-cut lung slice (PCLS) model was used to to investigate further the pharmacological properties of ICI-735.

### Example 9

### Inhibition of Airway Contraction in Human and Murine Models of Allergen, Independent of Inflammatory Mediator Release

Airway hyper-responsiveness (AHR) and inflammation characterizes airway diseases such as asthma. The results presented herein demonstrate that the compounds of the invention can inhibit human small airway contraction to multiple agonists as well as modulating a well characterized murine model of allergen induced AHR.

Human precision-cut lung slices (PCLS) permit evaluation of pharmacological responses of novel drug candidates applied directly to their target tissue. Carbachol (CCh), a cholinergic agonist, causes up to 70% bronchoconstriction in a dose-dependent manner when added at concentrations of 10⁻⁸ to 10⁻⁵ M. ICI-735 induces relaxation of maximally contracted airway smooth muscle in the presence of 30 μM CCh. As a positive control, isoproterenol (10 μM) induces a comparable degree of relaxation.

Pretreatment of lung slices with C-027 (10⁻⁵ M) blocks CCh-induced bronchoconstriction. Reduced responsiveness in the presence of the long-acting 02Adr-agonist formoterol (0.3 nM), demonstrates suitability of the experimental system.

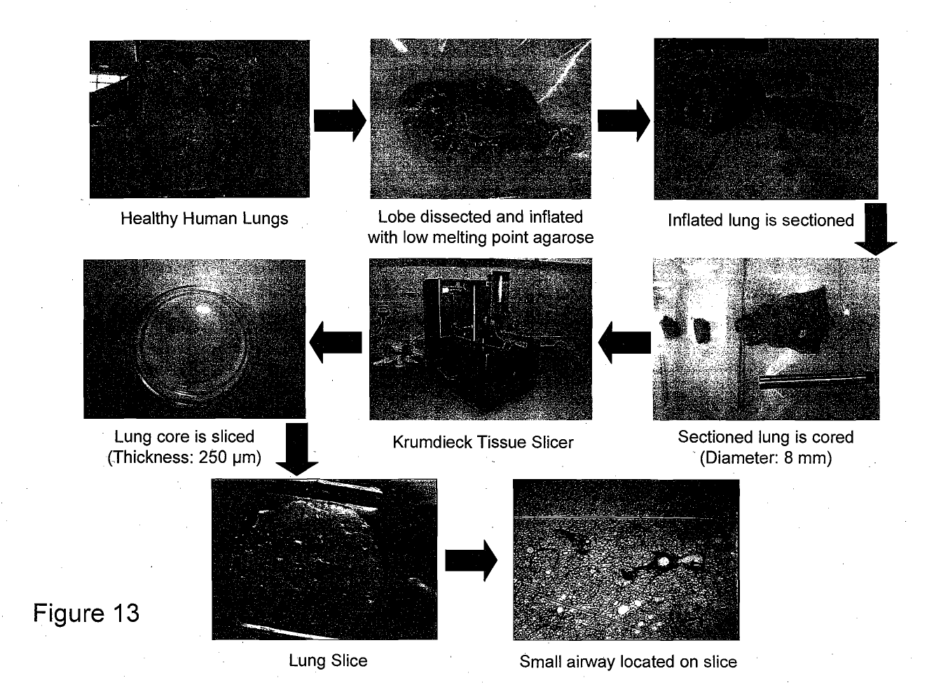

Human precision-cut lung slices (PCLS, 250 μm thick) containing a small airway were contracted to carbachol, histamine and passively sensitization to induce airway closure±pre-incubation with ICI-735 (10 μM; 2 hours); FIG. 13. For example, human lung slices containing a small airway were incubated overnight with either negative control IgG (4 μg/mL) or positive control IgE (4 μg/mL). The slices were tested as follows: 1) IgG+anti-IgE; 2) IgE+anti-IgE; 3) IgE+Dexamethasone (1 μM)+anti-IgE; and 4) IgE+ICI-735 (10 μM) +anti-IgE. Dexamethasone and ICI-735 were pre-incubated 2 hours prior to anti-IgE; anti-IgE is used to crosslink antibodies and induce mast cell degranulation, which is believed to release histamine and cause airway contract.

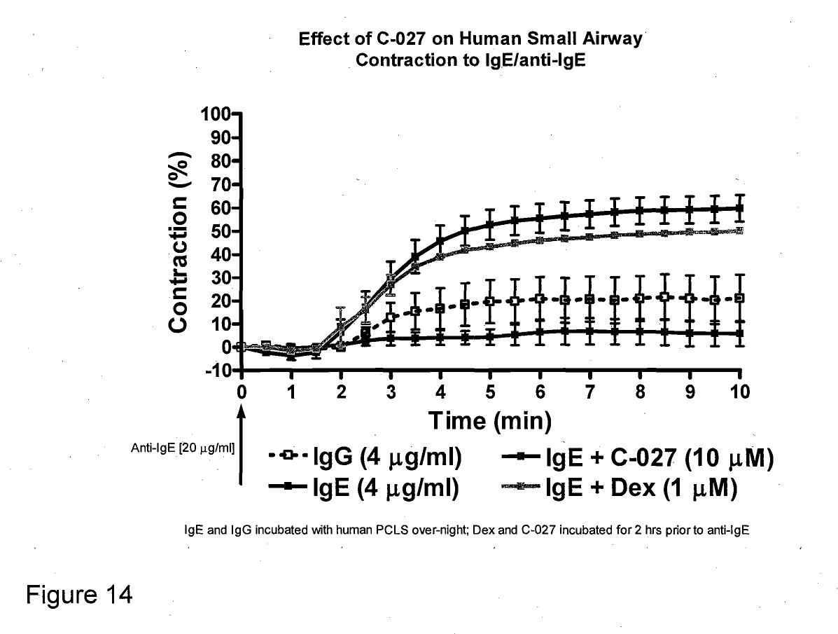

The results indicate that treatment of IgG+anti-IgE showed no contraction and showed IgE specificity. The treatment of IgE+anti-IgE showed about 60% contraction which is believed to be caused by mast cell degranulation. IgE+Dexamethasone treatment had no effect on passive sensitization because it is believed that 2 hours is too short of a time frame to induce genomic effects. Treatment of IgE+ICI-735 showed attenuation of mast cell degranulated induced airway contraction (FIG. 14).

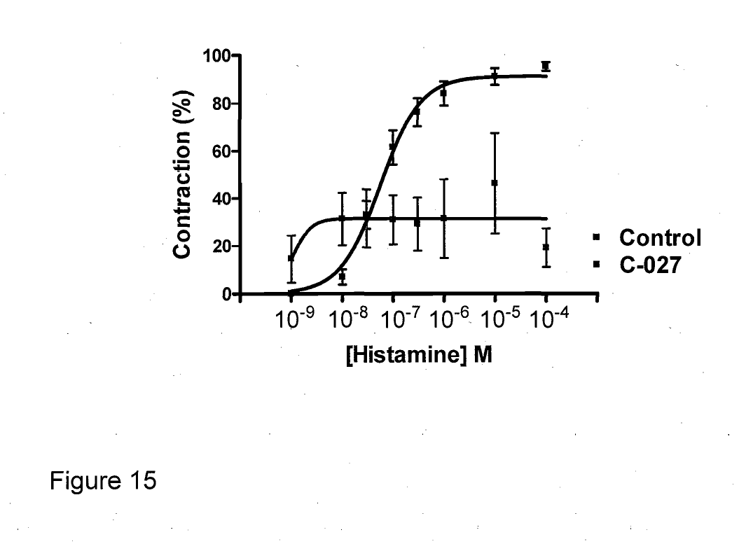

It was also observed that ICI-735 also blocked histamine induced contraction (FIG. 15).

It was also observed that ICI-735 diminishes IL-13-induced airway hyper-responsiveness. IL-13 is a Th2 cytokine capable of inducing in vivo all three major events characteristic of asthma: airway smooth muscle hyper-responsiveness to cholinergic agonists, eosinophilia, and mucous hyper-secretion. Overnight incubation of lung slices ex vivo with IL-13 (50 ng/mL) enhances contractile sensitivity of small airways to CCh in the PCLS model. Addition of ICI-735 to lung slices one hour prior to treatment with IL-13 diminishes IL-13-induced hyper-responsiveness to CCh in a C-027 dose-dependent manner.

It was also observed that ICI-735 blocks bronchoconstriction resulting from IgE-mediated mast cell degranulation. Human lung tissue contains multiple cell types, including mast cells, that localize in the region of airway smooth muscle, and that increase in number in individuals with asthma. Cross-linking of Fcε;R1 receptors on surfaces of mast cells leads to release of granules that contain smooth muscle contractile agonists, including histamine and leukotrienes. In lung slices sensitized by overnight incubation with IgE, then activated with anti-IgE as a cross-linking agent, bronchoconstriction commences approximately two minutes after addition of anti-IgE and generally reaches a maximum after five to six minutes. ICI-735 added prior to sensitization with IgE blocks bronchoconstriction in response to anti-IgE.

It was also observed that ICI-735 prevented histamine-induced bronchoconstriction. To test C-027 as a blocker of histamine-induced bronchoconstriction, lung slices were incubated for 2 hours with C-027, and small airway contraction was evaluated after addition of increasing concentrations of histamine. ICI-735 diminished sensitivity to histamine in a dose-responsive manner, but at the highest concentration tested (10 μM), does not completely prevent the histamine response.

Based on the results presented herein, ICI-735 prevents and reverses bronchoconstriction in response to cholinergic agents, both in normal and IL-13-induced hyper-responsive human lung tissue ex vivo. These observations reinforce findings in the murine allergic asthma model. ICI-735 appears to exert part of its effects by blocking histamine H-1 receptors. Anti-histamine activity alone, however, does not explain diminished responsiveness to methacholine. The observed weak or undetectable in vitro binding and functional activities toward M3 muscarinic, and AdrR2 receptors apparently rule out well-known anti-cholinergic and β2-agonist mechanisms.

Without wishing to be bound by any particular theory, it is believed that the compounds of the present invention can inhibit allergen-induced AHR independent of airway inflammation and inhibits human airway contraction to multiple agonists by a novel mechanism. The results presented herein demonstrate that the compositions of the invention leverages a novel mechanism that (i) prevents bronchoconstriction in response to histamine and cholinergic challenges; (ii) diminishes IL-13-induced hyper-responsiveness to cholinergics; and (iii) blocks bronchoconstriction secondary to IgE-mediated mast cell degranulation.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of rapidly treating a respiratory disease or disorder in a mammal, said method comprising administering to a mammal in need thereof a therapeutically effective amount of a compound, wherein said compound is capable of modulating activity of an immune cell and a muscle cell, further wherein said compound is formula I: 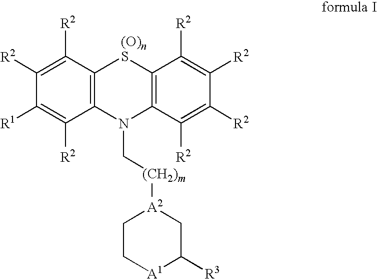
or a pharmaceutically acceptable salt, prodrug or solvate thereof, wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁ -C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is CH₂, N((CH₂)ₚNR⁷₂)₂, or NR⁴;
A² is CH or N;
provided that if A¹ is CH₂, then A² is N, and if A² is CH, then A¹ is NR⁴ or N((CH₂)ₚNR⁷₂)₂
R⁴ is H, (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;
R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.
R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;
R⁷ is independently selected at each occurrence from the group consisting of hydrogen and (C₁-C₆)alkyl;
R⁸ is 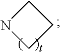
m is independently at each occurrence 1, 2, or 3;
n is 0, 1, or 2;
p is independently at each occurrence 2 or 3;
q is independently at each occurrence 1 or 2; and
t is 1, 2 or 3.

2. The method of claim 1, wherein said mammal is a human.

3. The method of claim 1, wherein said respiratory disease or condition is asthma.

4. The method of claim 1, wherein said compound is administered to the mammal orally, parenterally, intravascularly, intranasally, or intrabronchially.

5. The method of claim 1, wherein said administering comprises delivery via a mist route selected from the group consisting of aerosol inhalation, dry powder inhalation, liquid inhalation, and liquid instillation.

6. The method of claim 1, wherein the administered compound does not substantially modulate central nervous system function of the mammal.

7. The method of claim 1, wherein the administered compound does not substantially cross the blood-brain barrier of the mammal.

8. The method of claim 1, wherein said compound inhibits proliferation of an immune cell associated with said respiratory disease or condition when said compound binds to at least 5-HT1B receptor and 5-HT7 receptor on said immune cell.

9. The method of claim 8, wherein said immune cell is selected from the group consisting of a T cell, a B cell, a natural killer cell, a dendritic cell, and a macrophage, a monocyte, a neutrophil, a eosinophil, and a basophil.

10. The method of claim 1, wherein said compound is administered in combination with a therapeutic agent, wherein said therapeutic agent is an asthma/allergy medicament.

11. The method of claim 10, wherein said asthma/allergy medicament is selected from the group consisting of bronchodilator/beta-2 agonists, xanthanines, protease inhibitors, anti-histamines, steroids, prostaglandin inducers, immunomodulators, down-regulators of IgE.

12. The method of claim 11, wherein said therapeutic agent is administered simultaneously, prior to, or after administration of said compound.

13. The method of claim 1, wherein said muscle cell is selected from the group consisting of smooth muscle cell and cardiac muscle cell.

14. The method of claim 1, wherein said compound is administered at a dose of about 0.01 mg/kg parenterally.

15. The method of claim 1, wherein said compound is administered at a dose of about 1 mg/kg parenterally.

16. The method of claim 1, wherein said compound is administered at a dose of about 0.1 mg/kg orally.

17. The method of claim 1, wherein said compound is administered at a dose of about 10 mg/kg orally.

18. The method of claim 10, wherein said asthma/allergy medicament is selected from the group consisting of a β₂-adrenoceptor agonist, an adrenergic agonist, a methylxanthine, an antihistamine, a prostaglandin inducer, an inhaled glucocorticoid, a systemic glucocorticoid, an immunomodulator, a leukotriene modifier, an IgE blocker, a mast cell stabilizer, an anticholinergic, a methotrexate, a PDE-4 inhibitor, a bronchodilator/beta-2 agonist, a K+ channel opener, a VLA-4 antagonist, a neurokin antagonist, a TXA2 synthesis inhibitor, a xanthanine, an arachidonic acid antagonist, a 5 lipoxygenase inhibitor, a thromboxin A2 receptor antagonist, a thromboxane A2 antagonist, an inhibitor of 5-lipox activation protein, and a protease inhibitor.

19. The method of claim 18, wherein said β₂-adrenoceptor agonist is a long-acting β₂-adrenoceptor agonist.

20. The method of claim 18, wherein said β₂-adrenoceptor agonist is a short-acting β₂-adrenoceptor agonist.
